(19) United States
(12) Patent Application Publication
(10) Pub. No.: US 2018/0016642 A1
(43) Pub. Date: Jan. 18, 2018

(54) METHODS FOR ASSESSING THE RISK OF DISEASE OCCURRENCE OR RECURRENCE USING EXPRESSION LEVEL AND SEQUENCE VARIANT INFORMATION

(71) Applicant: VERACYTE, INC., South San Francisco, CA (US)

(72) Inventors: Giulia C. KENNEDY, San Francisco, CA (US); Moraima PAGAN, San Francisco, CA (US); Chu-Fang LIN, South San Francisco, CA (US); Jing HUANG, South San Francisco, CA (US); P. Sean WALSH, South San Francisco, CA (US); Hajime MATSUZAKI, Cupertino, CA (US); Kevin TRAVERS, South San Francisco, CA (US); Su Yeon KIM, South San Francisco, CA (US)

(21) Appl. No.: 15/694,157

(22) Filed: Sep. 1, 2017

Related U.S. Application Data
(63) Continuation of application No. PCT/US2016/020583, filed on Mar. 3, 2016.
(60) Provisional application No. 62/128,463, filed on Mar. 4, 2015.
(60) Provisional application No. 62/128,469, filed on Mar. 4, 2015.
(60) Provisional application No. 62/238,893, filed on Oct. 8, 2015.

(51) Int. Cl. C12Q 1/68; G06F 19/20; G06F 19/18; G06F 19/00

(52) U.S. Cl. CPC C12Q 1/6886; G06F 19/3431; G06F 19/20; G06F 19/18; C12Q 2600/158; C12Q 2600/156; C12Q 2600/118

(57) ABSTRACT

Provided herein are methods, systems and kits for stratification of risk of disease occurrence of a sample obtained from a subject by combining two or more feature spaces to improve individualization of subject management.

### CROSS REFERENCE

This application claims priority to U.S. provisional application 62/128,463, filed on Mar. 4, 2015, U.S. provisional application 62/128,469, filed on Mar. 4, 2015, and U.S. provisional application 62/238,893, filed on Oct. 8, 2015, each of which is entirely incorporated herein by reference.

### BACKGROUND

A risk adapted approach to a disease therapy, such as thyroid cancer therapy, may minimize the risk of disease occurrence, in addition to improving disease specific survival. Currently, this risk adapted approach to initial subject management is based in large part upon post-operative classification of subjects either as high, intermediate or low risk of disease recurrence utilizing the 2009 American Thyroid Association (ATA) staging system. While this anatomic staging system has proven clinically useful, it cannot be accurately assessed prior to an invasive thyroidectomy, and it does not include any molecular predictors of disease outcome.

### SUMMARY

Provided herein are various methods for assessing or stratifying risk of disease occurrence and/or recurrence. Transcriptional data obtained during pre-diagnostic or diagnostic evaluation, such as fine needle aspiration (FNA), can improve the pre-operative prediction of risk occurrence of a disease such as thyroid cancer, and can provide further individualization of subject therapy and treatment. Methods of the present disclosure may provide an assessment with respect to a risk of occurrence and/or recurrence of a disease in a relatively noninvasive manner and using low sample volumes.

An aspect of the present disclosure provides a method for evaluating a tissue sample of a subject to determine a risk of occurrence of disease in the subject. The method comprises (a) obtaining an expression level corresponding to each one or more genes of a first set of genes in a nucleic acid sample in a needle aspirate sample obtained from the subject, which first set of genes is associated with the risk of occurrence of disease in the subject; (b) determining a presence of a nucleic acid sequence corresponding to each of one or more genes of a second set of genes in the nucleic acid sample, which second set of genes is associated with the risk of occurrence of disease in the subject; (c) separately comparing to controls (i) the expression level obtained in (a) and (ii) the nucleic acid sequence obtained in (b) to provide comparisons of the expression level and the nucleic acid sequence to the controls, wherein a comparison of the nucleic acid sequence to a reference sequence among the controls is indicative of a presence of one or more sequence variants with respect to a given gene of the second set of genes; and (d) using a computer processor that is programmed with a trained algorithm to (i) analyze the comparisons and (ii) determine the risk of occurrence of the disease based on the comparisons.

In some embodiments, the needle aspirate sample is a fine needle aspirate sample. In some embodiments, the disease is cancer. In some embodiments, the method further comprises, prior to (a), obtaining the needle aspirate sample from the subject. In some embodiments, the method further comprises, prior to (a), determining the expression level from the nucleic acid sample in the needle aspirate sample. In some embodiments, the method further comprises, prior to (b), determining the nucleic acid sequence from the nucleic acid sample in the needle aspirate sample. In some embodiments, the method further comprises comparing the nucleic acid sequence to the reference sequence to identify the one or more sequence variants. In some embodiments, the reference sequence is a housekeeping gene from the subject. In some embodiments, the one or more genes in the first set or second set of genes include a plurality of genes.

In some embodiments, the needle aspirate sample has been found to be cytologically ambiguous or suspicious. In some embodiments, the needle aspirate sample has a volume that is about 1 microliter or less. In some embodiments, the needle aspirate sample has an RNA Integrity Number (RIN) value of about 9.0 or less. In some embodiments, RNA purified from a needle aspirate sample has an RNA RIN value of about 9.0 or less. In some embodiments, the needle aspirate sample has an RIN value of about 6.0 or less. In some embodiments, the RNA sample has an RIN value of about 6.0 or less.

In some embodiments, the risk of occurrence of the disease includes a risk of recurrence of the disease in the subject. In some embodiments, the risk of occurrence of the cancer includes a risk of metastasis in the subject. In some embodiments, the risk of occurrence of cancer includes a risk of accelerated disease progression. In some embodiments, the risk of occurrence of cancer includes a risk of therapeutic failure.

In some embodiments, the trained algorithm is trained employing tissue samples from at least 25 or at least 100 subjects having been diagnosed with the disease. In some embodiments, the trained algorithm is trained employing tissue samples from at least 200 subjects having been diagnosed with the disease.

In some embodiments, (d) occurs pre-operatively. In some embodiments, (d) occurs prior to the subject having a positive disease diagnosis. In some embodiments, (d) further comprises stratifying the risk of occurrence into a low risk of occurrence or a medium-to-high risk of occurrence, wherein the low risk of occurrence has a probability of occurrence between about 50% and about 80% and wherein the medium-to-high risk of occurrence has a probability of occurrence between about 80% and 100%.

In some embodiments, the method further comprises applying one or more filters, one or more wrappers, one or more embedded protocols, or any combination thereof to the comparisons. In some embodiments, the one or more filters are applied to the comparisons. In some embodiments, the one or more filters comprise a t-test, an analysis of variance (ANOVA) analysis, a Bayesian framework, a Gamma distribution, a Wilcoxon rank sum test, between-within class sum of squares test, a rank products method, a random permutation method, a threshold number of misclassification (TNoM), a bivariate method, a correlation based feature selection (CFS) method, a minimum redundancy maximum relevance (MRMR) method, a Markov blanket filter method, an uncorrelated shrunken centroid method, or any combination thereof. In some embodiments, the one or more sequence variants comprise one or more of a point mutation, a fusion gene, a substitution, a deletion, an insertion, an inversion, a conversion, a translocation, or any combination thereof. In some embodiments, the one or more point mutations are from about 5 to about 4000 point mutations. In some embodiments, the one or more fusion genes are at least two fusion genes.

In some embodiments, the stratifying has an accuracy of about 80%. In some embodiments, the stratifying has a specificity of about 80%. In some embodiments, the one or more genes of the first or second set is less than about 15 genes or less than about 10 genes. In some embodiments, the one or more genes of the first or second set is less than about 75 genes. In some embodiments, the one or more genes of the first or second set is between about 50 and about 400 genes.

In some embodiments, the obtaining in (b) comprises sequencing a nucleic acid sample in the needle aspirate sample to obtain the nucleic acid sequence. In some embodiments, the sequencing comprises enriching for the one or more genes of a second set of genes, or variants thereof. In some embodiments, (a) comprises using a microarray with probes that are selective for the one or more genes of the first set of genes. In some embodiments, (a) comprises using a targeted sequencing platform (such as Ion Torrent Ampliseq, or Illumina TruSeq Custom Amplicon).

In some embodiments, the tissue sample is a thyroid tissue sample. In some embodiments, the first and second sets of genes comprise COL1A1, THBS2, or any combination thereof. In some embodiments, the second set of genes comprise EPHA3, COL1A1, EHF, RAPGEF5, PRICKLE1, TMEM92, ROBO1, C6orf136, SPAG4, GALNT15, LUM, NCAM2, NUP210L, NR2F1, THBS2, PSORS1C1, or any combination thereof. In some embodiments, the first set of genes comprises COL1A1, TMEM92, C1orf87, SPAG4, EHF, COL3A1, GALNT15, NUP210L, PDZRN3, C6orf136, NA, NRXN3, COL6A3, RAPGEF5, PRICKLE1, LUM, ROBO1, BGN, AC019117.2, PRSS3P1, or any combination thereof. In some embodiments, the second set of genes comprises EPHA3, COL1A1, EHF, RAPGEF5, PRICKLE1, TMEM92, ROBO1, C6orf136, SPAG4, GALNT15, LUM, NCAM2, SYNPO2, NUP210L, AMZ1, NR2F1, THBS2, PSORS1C1, FTH1P24, or any combination thereof. In some embodiments, the second set of genes comprises AKAP9, SPRY3, SPRY3, CAMKK2, COL1A1, FITM2, COX6C, VSIG10L, CYC1, KDM1B, MAPK15, ARSG, PAXIP1, DAAM1, AVL9, DMGDH, HLA-DQA1, HLA-DQB1, HLA-DRA, HLA-DRB5, HLA-H, IRF1, MGAT1, P2RX1, PLEK, CCDC93, PPP1R12C, SLC41A3, METTL3, CCAR2, PTPRE, SRL, SLC30A5, BMP4, ZNF133, ICE2, DCAKD, TMX1, TNFSF12, PER2, MCM3AP, or any combination thereof.

In some embodiments, the first set of genes and the second set of genes are different. In some embodiments, the method further comprises identifying new genetic biomarkers of the disease.

In some embodiments, the obtaining in (a) comprises assaying for the expression level corresponding to each of the one or more genes. In some embodiments, the assaying comprises array hybridization, nucleic acid sequencing or nucleic acid amplification using markers that are selected for each of the one or more genes. In some embodiments, the markers are primers that are selected for each of the one or more genes.

In some embodiments, the assaying comprises reverse transcription polymerase chain reaction (PCR). In some embodiments, the determining comprises assaying for each of the one or more genes of the second set of genes in the nucleic acid sample. In some embodiments, the assaying comprises array hybridization, nucleic acid sequencing or nucleic acid amplification using markers that are selected for each of the one or more genes. In some embodiments, the markers are primers that are selected for each of the one or more genes. In some embodiments, the assaying comprises reverse transcription polymerase chain reaction (PCR).

Another aspect of the present disclosure provides a computer-readable medium (e.g., memory) comprising machine-executable code that, upon execution by one or more computer processors, implements any of the methods above or elsewhere herein.

Another aspect of the present disclosure provides a computer system comprising one or more computer processors and a computer-readable medium coupled thereto. The computer-readable medium may comprise machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a sample cohort of cytology data and expert histopathology data stratified into low risk and medium-to-high risk of occurrence of cancer;

FIG. 2 shows histopathology risk features and the number and percent of samples for each feature;

FIG. 3 shows cross validation of true positive rates plotted against false positive rates;

FIG. 4 shows classification performance data plotting predictive values against prevalence of medium-to-high risk;

FIG. 5 shows classification performance data across low risk and medium-to-high risk groups;

FIG. 6 shows an example list of genes associated with a risk of occurrence of thyroid cancer based on gene expression level data;

FIG. 7 shows an example list of genes associated with a risk of occurrence of thyroid cancer based on gene expression level data obtained from ribonucleic acid (RNA) sequencing;

FIG. 8 shows an example list of genes associated with a risk of occurrence of thyroid cancer based on sequence variant data;

FIG. 9 shows a computer control system that is programmed or otherwise configured to implement methods provided herein;

FIG. 10 shows a flow diagram of determining accurate training labels;

FIG. 11A shows cross validation of true positive rates plotted against false positive rates;

FIG. 11B shows classification performance data across intermediate/high risk and low risk groups;

FIG. 12 shows an example list of genes of variants selected by the classifier in each fold;

FIG. 13 shows an example list of genes of counts selected 8 to 10 times by the classifier in 10 folds;

FIG. 14 shows a table of five point mutation panels and fusion pairs;

FIG. 15 shows a graph of test performance specificity and sensitivity across five panels of mutations and fusion pairs;

FIG. 16 shows a table of mutation performance of panel 3 in FIGS. 14 and 15 by cytology);

FIG. 17 shows a graph of test performance specificity and sensitivity across five panels of mutations and fusion pairs;

FIG. 18A shows a graphical representation; FIG. 18B shows a table representation of mutation frequency of a Clinical Laboratory Improvement Amendments (CLIA) fine needle aspirate (FNA) sample;

FIG. 19A shows a graphical representation; FIG. 19B shows a table representation of mutation frequency of a FNA sample; and

FIG. 20A shows a graphical representation; FIG. 20B shows a table representation of mutation frequency of a tissue sample.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about 1, 2, 5, 10, 20, 30, 40, 50, 60, 65, 70, 75, or about 80 years of age. The subject may have or be suspected of having a disease, such as cancer. The subject may be a patient, such as a patient being treated for a disease, such as a cancer patient. The subject may be predisposed to a risk of developing a disease such as cancer. The subject may be in remission from a disease, such as a cancer patient. The subject may be healthy.

The term “disease,” as used herein, generally refers to any abnormal or pathologic condition that affects a subject. Examples of a disease include cancer, such as, for example, thyroid cancer, parathyroid cancer, lung cancer, skin cancer, and others. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein.

The term “risk of occurrence of disease,” as defined herein, generally refers to a risk or probability associated with the occurrence of a disease in a subject. A risk of occurrence can include a first occurrence of disease in a subject or can include subsequent occurrences, such as a second, third, fourth, or subsequent occurrence. A risk of occurrence of disease can include a) a risk of developing the disease for a first time, b) a risk of relapse or of developing the disease again, c) a risk of developing the disease in the future, d) a risk of being predisposed to developing the disease in the subject's lifetime, or e) a risk of being predisposed to developing the disease as an infant, adolescent, or adult. A risk of occurrence of a disease, such as cancer, can include a risk of the cancer becoming metastatic. A risk of occurrence of a disease such as cancer can include a risk of occurrence of a stage I cancer, a stage II cancer, a stage III cancer, or a stage IV cancer. Risk of occurrence of cancer can include a risk for a blood cancer, tissue cancer (e.g., a tumor), or a cancer becoming metastatic to one or more organ sites from other sites.

The term “sequence variant,” “sequence variation,” “sequence alteration” or “allelic variant,” as used herein, generally refer to a specific change or variation in relation to a reference sequence, such as a genomic deoxyribonucleic acid (DNA) reference sequence, a coding DNA reference sequence, or a protein reference sequence, or others. The reference DNA sequence can be obtained from a reference database. A sequence variant may affect function. A sequence variant may not affect function. A sequence variant can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a sequence variation can include a substitution, a deletion, an insertion, an inversion, or a conversion in one or more nucleotides or one or more amino acids. A sequence variant may be a point mutation. A sequence variant may be a fusion gene. A fusion pair or a fusion gene may result from a sequence variant, such as a translocation, an interstitial deletion, a chromosomal inversion, or any combination thereof. A sequence variation can constitute variability in the number of repeated sequences, such as triplications, quadruplications, or others. For example, a sequence variation can be an increase or a decrease in a copy number associated with a given sequence (i.e., copy number variation, or CNV). A sequence variation can include two or more sequence changes in different alleles or two or more sequence changes in one allele. A sequence variation can include two different nucleotides at one position in one allele, such as a mosaic. A sequence variation can include two different nucleotides at one position in one allele, such as a chimeric. A sequence variant may be present in a malignant tissue. A sequence variant may be present in a benign tissue. Absence of a variant may indicate that a tissue or sample is benign. As an alternative, absence of a variant may not indicate that a tissue or sample is benign.

The term “mutation panel,” as used herein, generally refers to a panel designating a specified number of genomic sites and fusion pairs that are to be detected (or interrogated) with a risk classifier. For example, a mutation panel may comprise 9 genomic sites and 3 fusion pairs to be interrogated. Increasing the sensitivity of a risk classifier by increasing the number of point mutations and fusion pairs detected may decrease the sensitivity of a risk classifier.

A mutation panel may comprise one or more genomic sites and one or more fusion pairs. A mutation panel may comprise more than about 1, 2, 3, 4, or 5 genomic sites. A mutation panel may comprise more than about 15 genomic sites. A mutation panel may comprise more than about 100 genomic sites. A mutation panel may comprise more than about 200 genomic sites. A mutation panel may comprise more than about 500 genomic sites. A mutation panel may comprise more than about 1000 genomic sites. A mutation panel may comprise more than about 2000 genomic sites. A mutation panel may comprise more than about 3000 genomic sites. A mutation panel may comprise more than about 1 or 2 fusion pairs. A mutation panel may comprise more than about 5 fusion pairs. A mutation panel may comprise more than about 10 fusion pairs. A mutation panel may comprise more than about 15 fusion pairs. A mutation panel may comprise more than about 20 fusion pairs. A mutation panel may comprise more than about 25 fusion pairs.

The term “disease diagnostic,” as used herein, generally refers to diagnosing or screening for a disease, to stratify a risk of occurrence of a disease, to monitor progression or remission of a disease, to formulate a treatment regime for the disease, or any combination thereof. A disease diagnostic can include a) obtaining information from one or more tissue samples from a subject, b) making a determination about whether the subject has a particular disease based on the information or tissue sample obtained, c) stratifying the risk of occurrence of the disease in the subject, d) confirming whether a subject has the disease, is developing the disease, or is in disease remission, or any combination thereof. The disease diagnostic may inform a particular treatment or therapeutic intervention for the disease. The disease diagnostic may also provide a score indicating for example, the severity or grade of a disease such as cancer, or the likelihood of an accurate diagnosis, such as via a p-value, a corrected p-value, or a statistical confidence indicator. The disease diagnostic may also indicate a particular type of a disease. For example, a disease diagnostic for thyroid cancer may indicate a subtype such as follicular adenoma (FA), nodular hyperplasia (NHP), lymphocytic thyroiditis (LCT), Hürthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), anaplastic thyroid carcinoma (ATC), renal carcinoma (RCC), breast carcinoma (BCA), melanoma (MMN), B cell lymphoma (BCL), parathyroid (PTA), or hyperplasia papillary carcinoma (HPC).

### Methods for Evaluating a Risk of Occurrence or Recurrence of a Disease

The present disclosure provides methods for evaluating a tissue sample of a subject to determine a risk of occurrence or recurrence of disease in the subject and in some cases to determine new genetic biomarkers of the disease. Such methods can comprise obtaining an expression level corresponding to each of one or more genes of a first set of genes in a nucleic acid sample obtained from the subject. In some cases, the expression level is obtained using a microarray with probes that are selective for the one or more genes of the first set of genes. The nucleic acid sample may be obtained by the subject or by another individual, such as a medical professional. The first set of genes may be associated with the risk of occurrence of disease in the subject. In some examples, the nucleic acid sample is obtained by FNA, surgery (e.g., surgical biopsy), or other approaches for obtaining a sample from the subject. The nucleic acid sample may be in a tissue sample (such as a thyroid tissue sample), a blood sample, or a fluid sample obtained from the subject. In an example, the nucleic acid sample may be included in an FNA sample obtained from the subject.

Next, a presence of a nucleic acid sequence corresponding to each of one or more genes of a second set of genes in the nucleic acid sample is determined. The second set of genes may be associated with the risk of occurrence of disease in the subject. In some examples, the presence of the sequence is determined by sequencing the nucleic acids in the FNA sample to obtain the nucleic acid sequence. The sequencing may also enrich for the one or more genes of a second set of genes, or variants thereof.

Next, the obtained expression level and the obtained nucleic acid sequence are compared to controls to provide comparisons of the expression level and the nucleic acid sequence to the controls. A comparison of the nucleic acid sequence to a reference sequence among the controls may be indicative of a presence of one or more sequence variants with respect to a given gene of the second set of genes. The reference sequence can be, for example, a housekeeping gene obtained from the subject.

Next, the comparisons are analyzed and the risk of occurrence or recurrence of the disease is determined based on the comparisons. In some examples, an algorithm implemented by one or more programmed computer processors is used to analyze the comparisons and determine the risk of occurrence or recurrence of the disease. The algorithm may be a trained algorithm (e.g., an algorithm that is trained on at least 10, 200, 100 or 500 reference samples). References samples may be obtained from subjects having been diagnosed with the disease or from healthy subjects.

In some examples, the expression level for each of the one or more genes of a first set of genes can be obtained by assaying for the expression level. In some examples, the presence of a nucleic acid sequence corresponding to each of the one or more genes of a second set of genes can by determined by assaying for each of the one or more genes. In such examples, assaying may comprise array hybridization, nucleic acid sequencing, nucleic acid amplification, or others. Assaying may comprise sequencing, such as DNA or RNA sequencing. Such sequencing may be by next generation (NextGen) sequencing. Assaying may comprise reverse transcription polymerase chain reaction (PCR). Assaying may utilize markers, such as primers, that are selected for each of the one or more genes of the first or second sets of genes.

Before obtaining the expression level corresponding to the one or more genes of the first set of genes, the sample may be obtained from the subject. The expression level of a plurality of genes of the nucleic acid sample may also be determined prior to obtaining the expression level corresponding to the one or more genes of the first set of genes. In some cases, before determining the presence of a nucleic acid sequence of the second set of genes, nucleic acid sequences of the plurality of genes in the sample can be determined.

In some examples, the disease is cancer, such as thyroid cancer, breast cancer or others. Determining a risk of occurrence or recurrence can also be determined in non-cancerous diseases such as a genetic disorder, a hyper-proliferative disorder or others.

The sample obtained from the subject may be cytologically ambiguous or suspicious (or indeterminate). In some cases, the sample may be suggestive of the presence of a disease. The volume of sample obtained from the subject may be small, such as about 100 microliters, 50 microliters, 10 microliters, 5 microliters, 1 microliter or less. The sample may comprise a low quantity or quality of polynucleotides, such as a tissue sample with degraded or partially degraded RNA. For example, an FNA sample may yield low quantity or quality of polynucleotides. In such examples, the RNA Integrity Number (RIN) value of the sample may be about 9.0 or less. In some examples, the RIN value may be about 6.0 or less.

The risk of occurrence of the disease may include a risk of a subsequent occurrence such as a second, third, fourth, or more subsequent occurrences. A risk of occurrence of disease can include one or more of a) a risk of developing the disease for a first time, b) a risk of relapse or of developing the disease again, c) a risk of developing the disease in the future, d) a risk of being predisposed to developing the disease in a subject's lifetime, e) a risk of being predisposed to developing the disease as an infant, adolescent, or adult. In cases where the disease is cancer, a risk of occurrence can include a risk of the cancer becoming metastatic.

A determination of risk can be completed pre-operatively, such as before a patient's surgery. A clinician may recommend that a patient be continued to be observed rather than recommending surgery, if the patient, for example, is determined to have a low-risk of papillary thyroid carcinoma. In some cases, a clinical is more likely to recommend a patient to have surgery, if the patient is determined to have a high-risk of papillary thyroid carcinoma. A determination can occur prior to the subject having a positive disease diagnosis, such as when a subject is suspected of having a disease or during a routine clinical procedure.

A determination of risk may further comprise stratifying the risk into a low risk of occurrence or a medium-to-high risk of occurrence. In some examples, the low risk may be a probability of occurrence between about 50% and about 80% and medium-to-high risk may be a probability of occurrence between about 80% and 100%.

Accurately stratifying the risk into low and medium-to-high risk groups can occur in about 80% of samples analyzed. Stratifying the risk can be accurately determined in about 50%, 60%, 70%, 75%, 80%, 85%, 90%, 95%, 96%, 97%, 98%, or about 99% of samples analyzed, including samples identified as cytologically ambiguous or suspicious. Stratifying the risk into low and medium-to-high risk groups can be at least about 80% specific. In some examples, the specificity of stratifying the risk can be about 50%, 60%, 70%, 75%, 80%, 85%, 90%, 95%, 96%, 97%, 98%, 99% or more, including samples identified as cytologically ambiguous or suspicious.

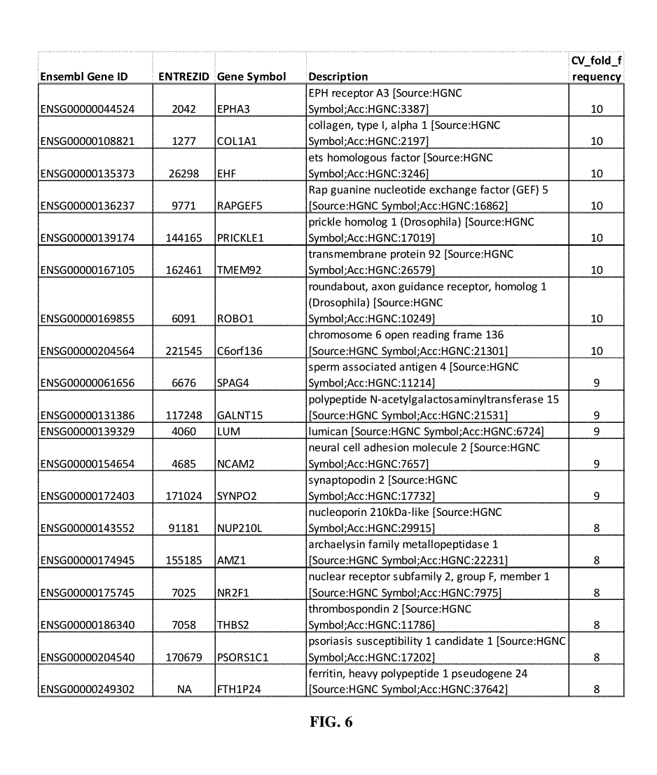

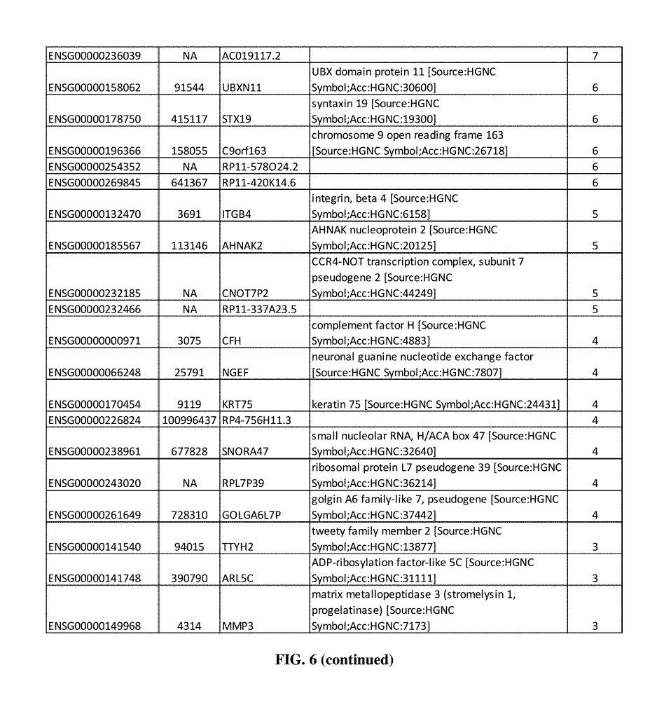

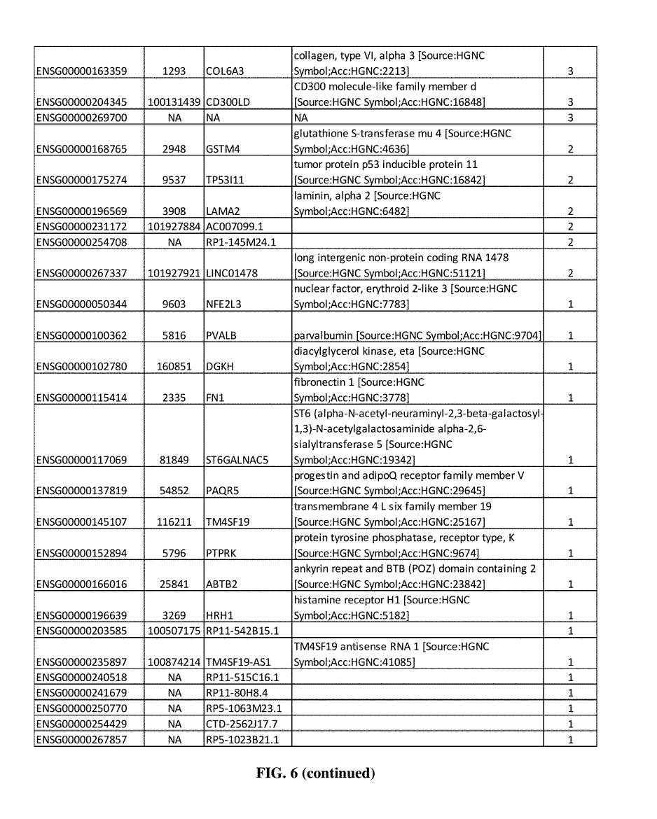

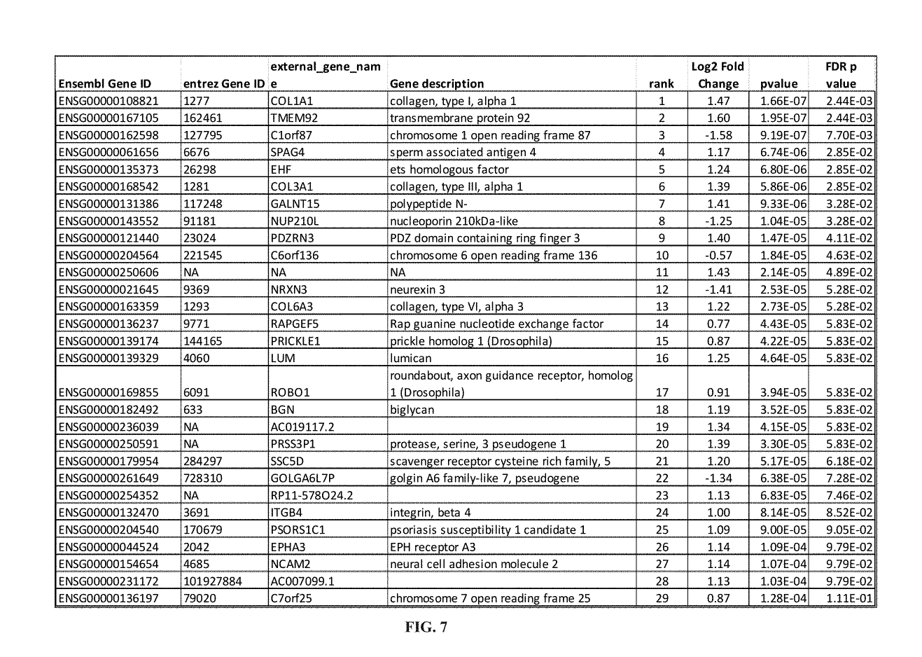

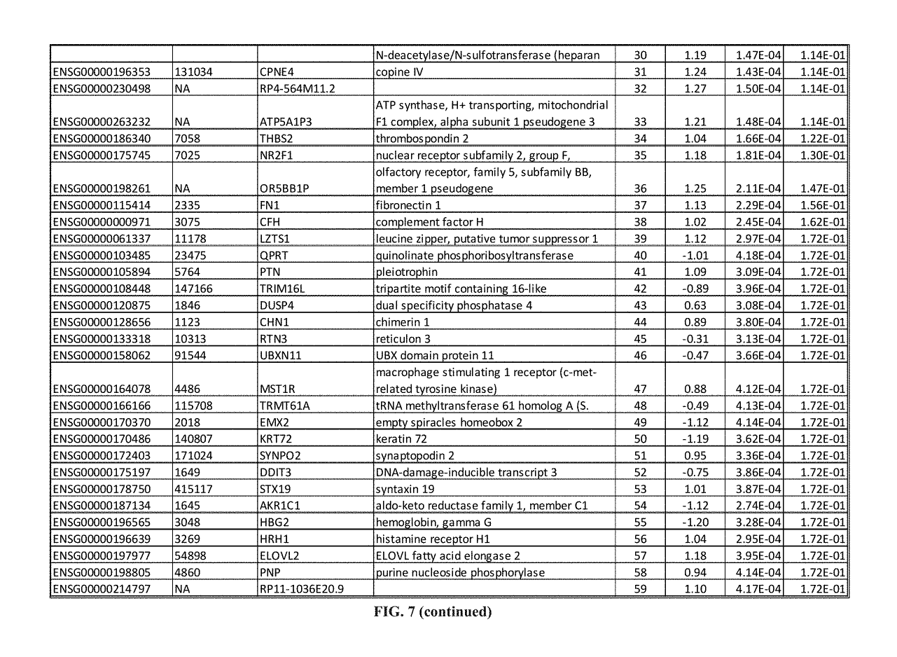

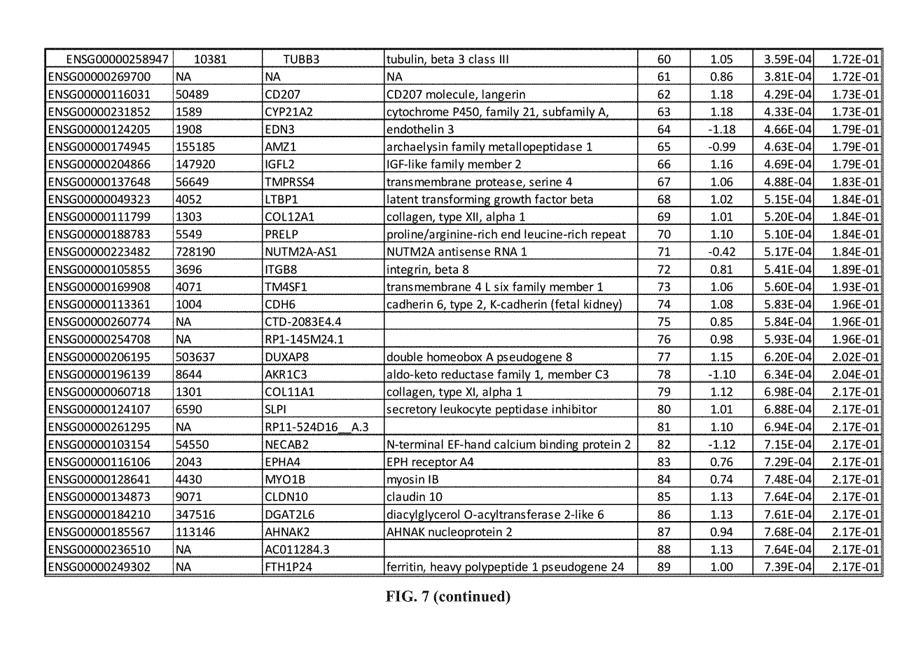

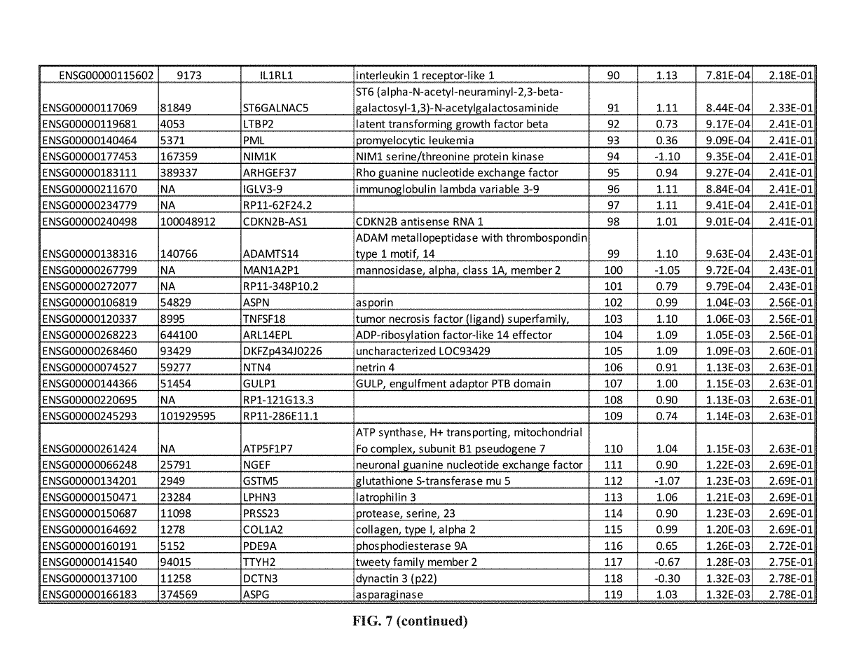

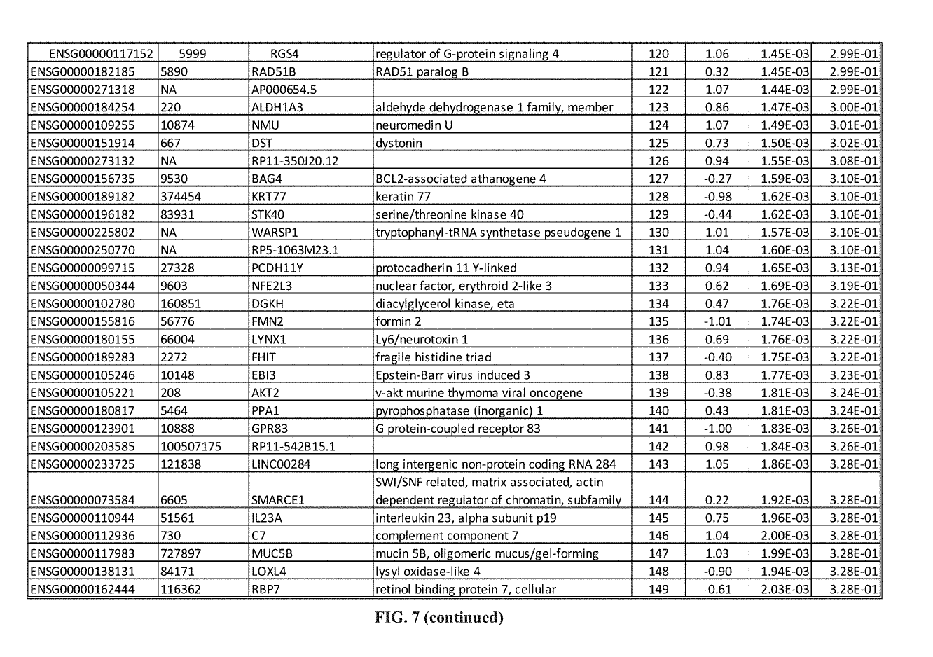

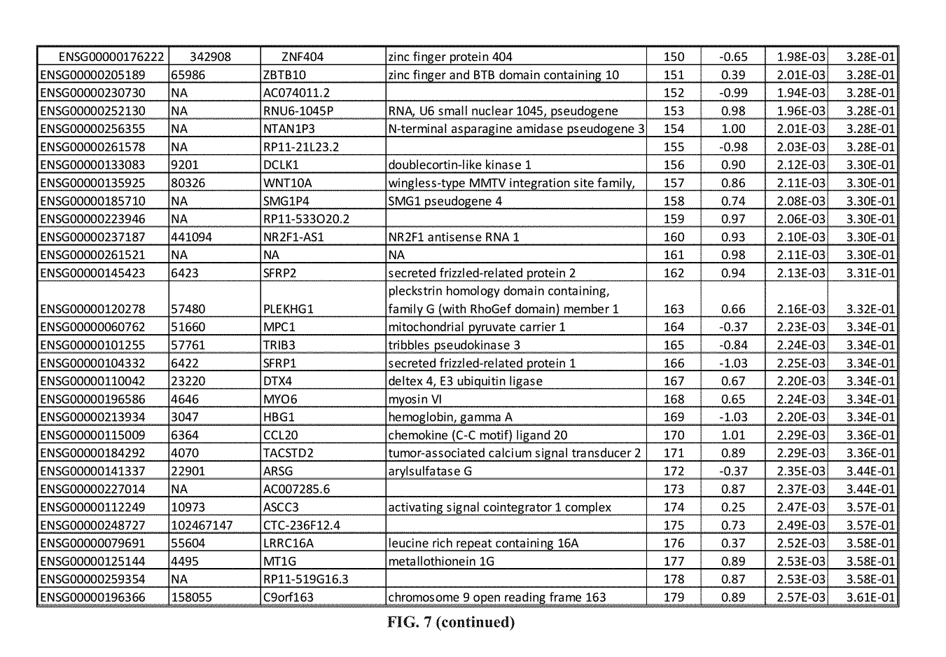

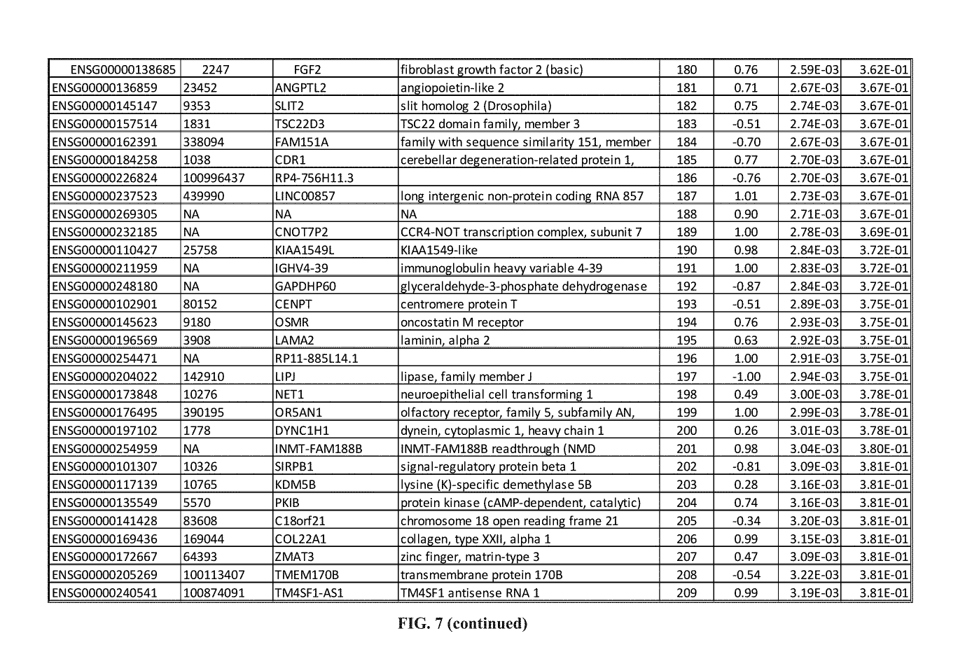

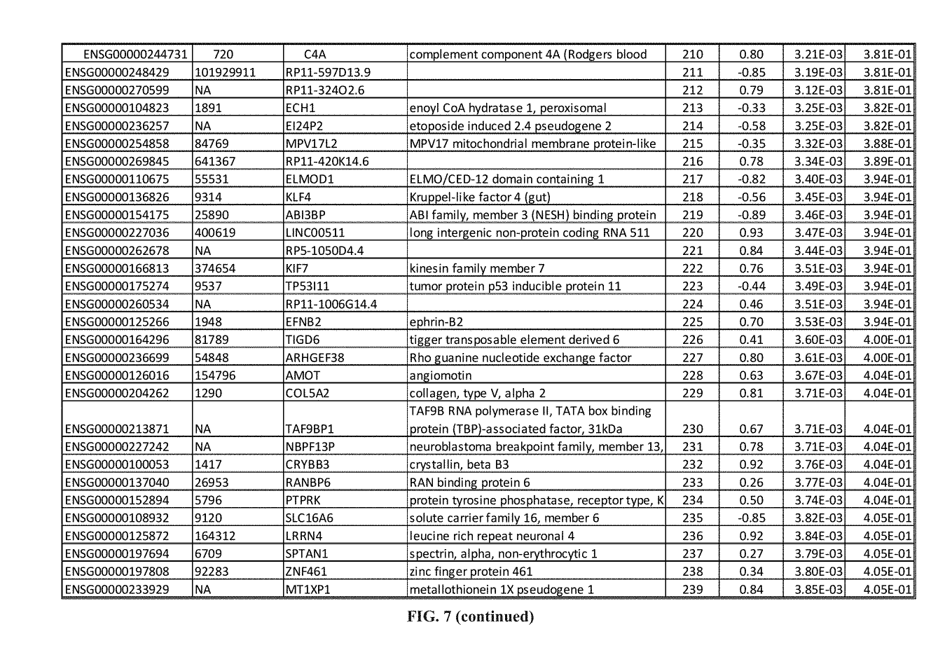

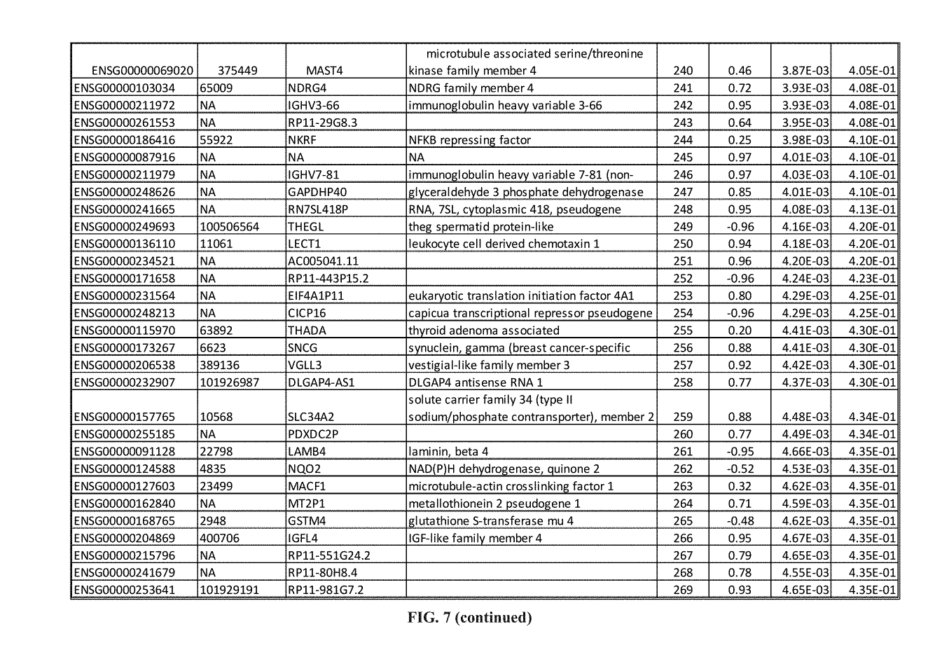

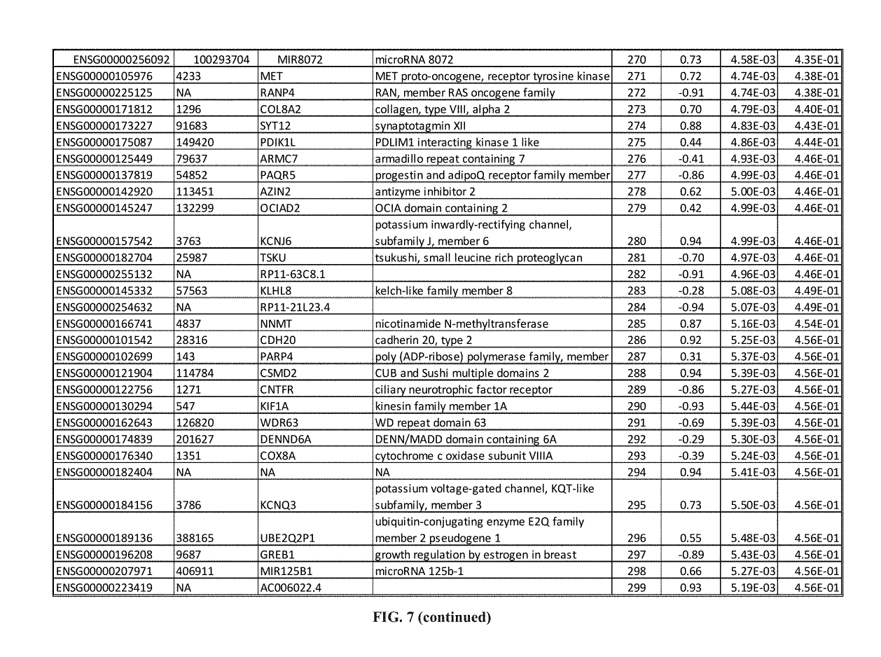

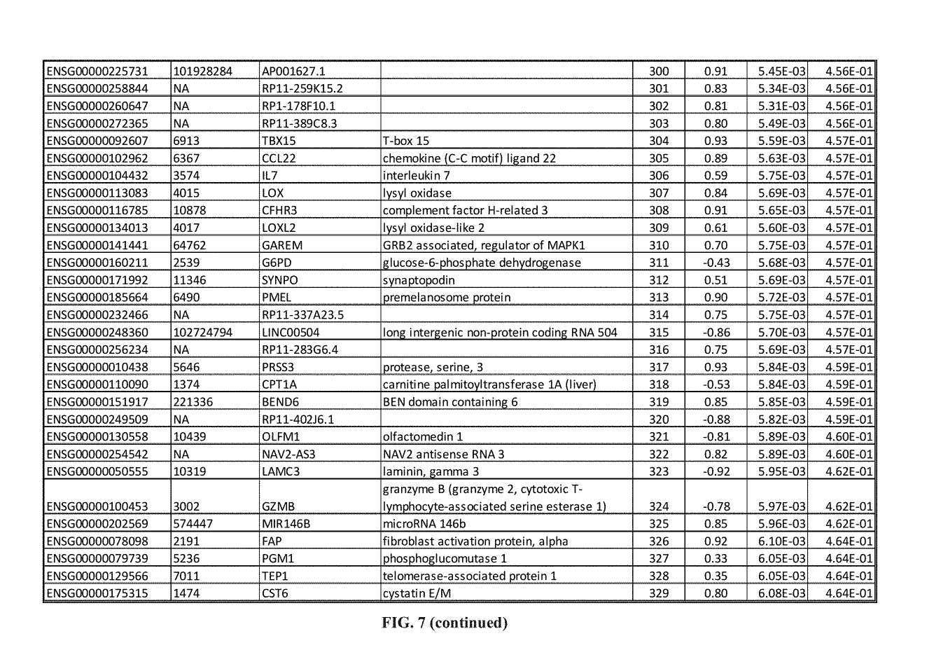

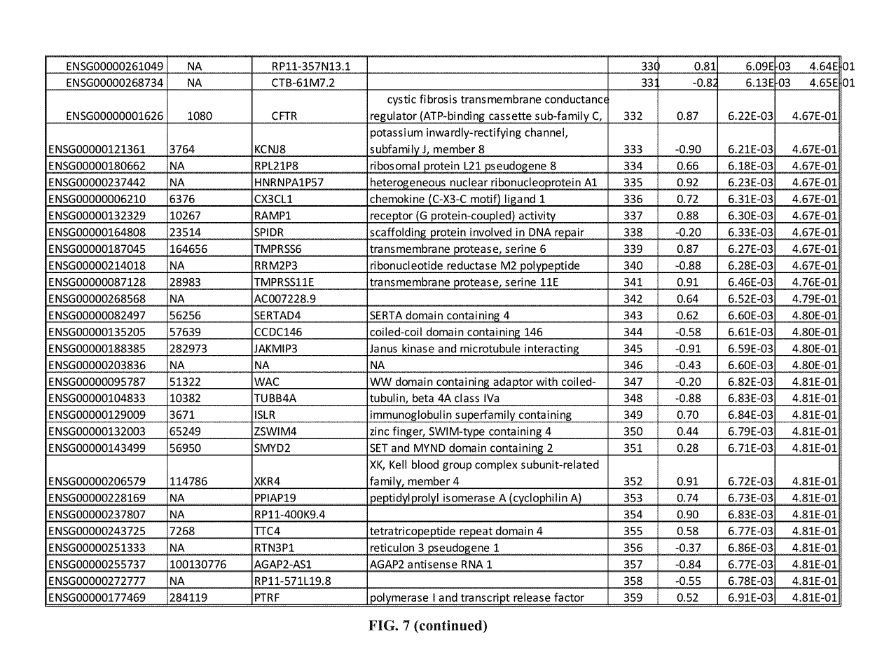

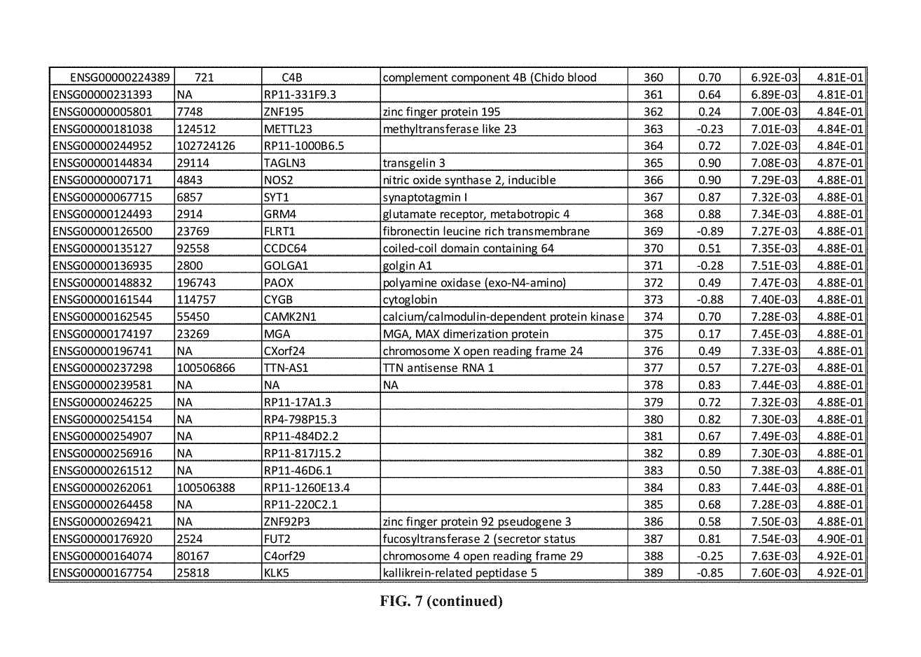

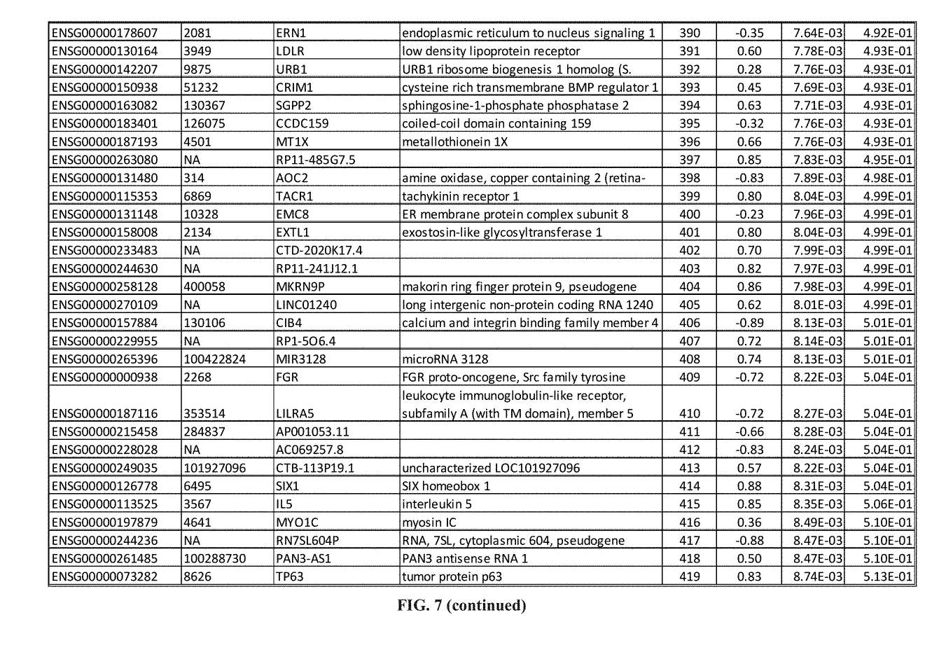

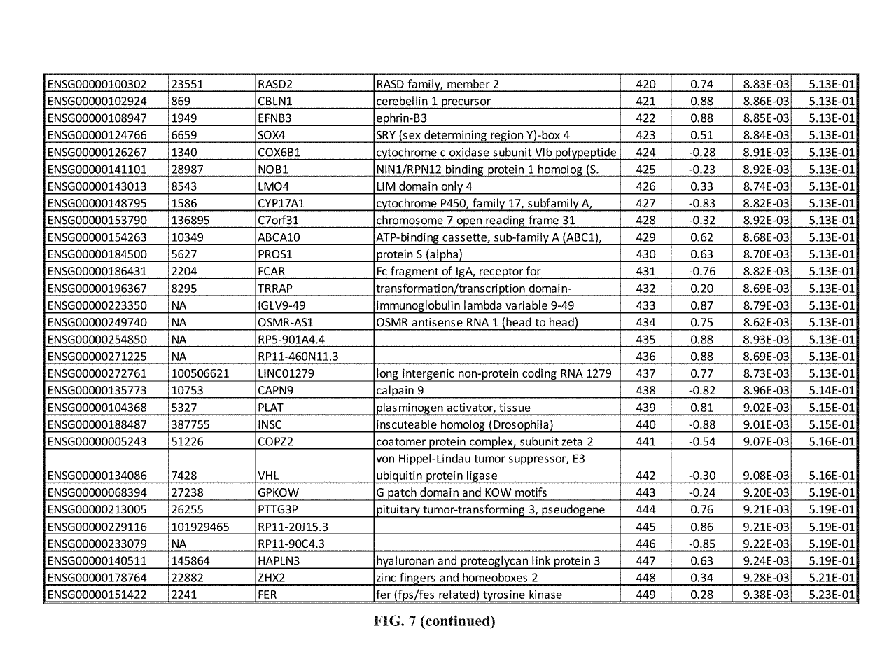

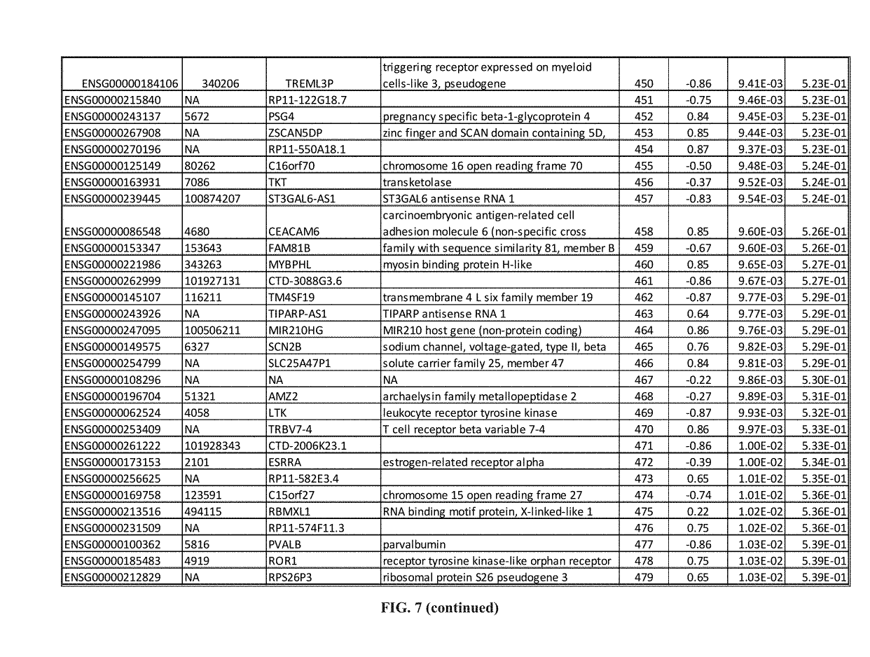

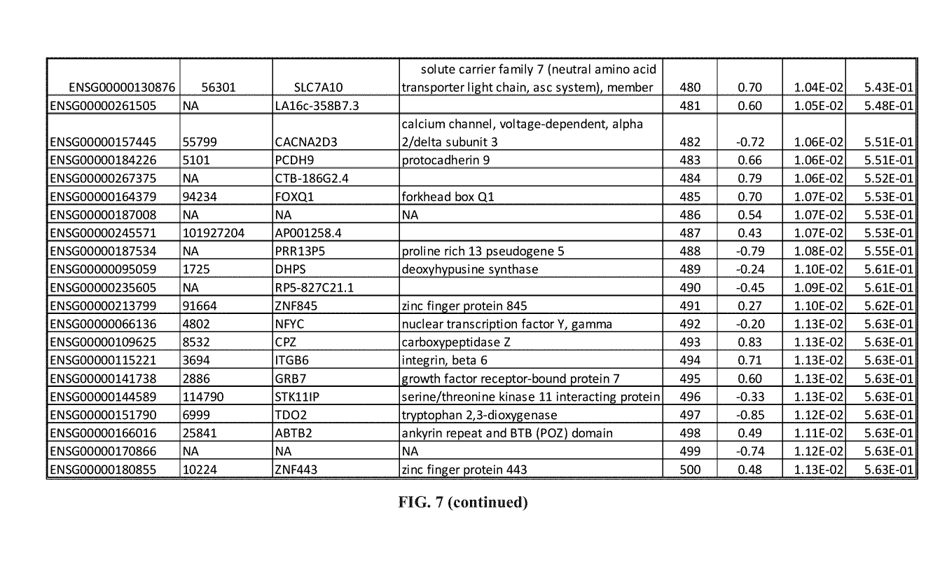

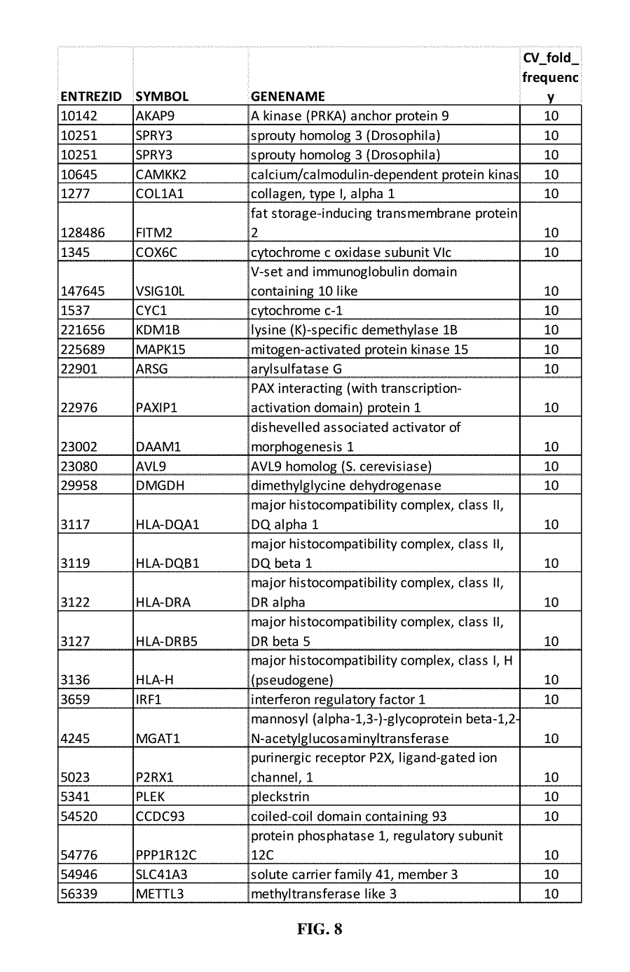

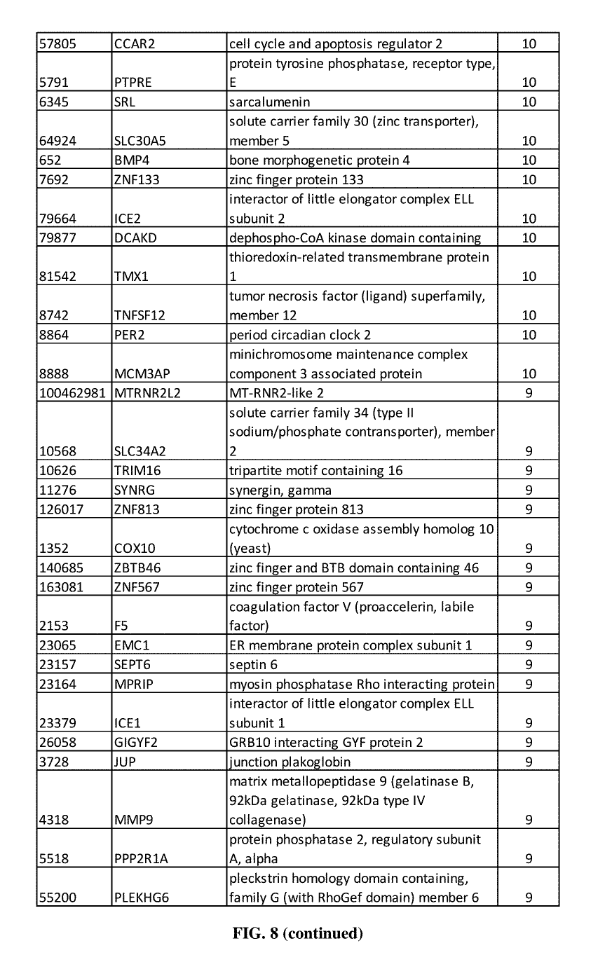

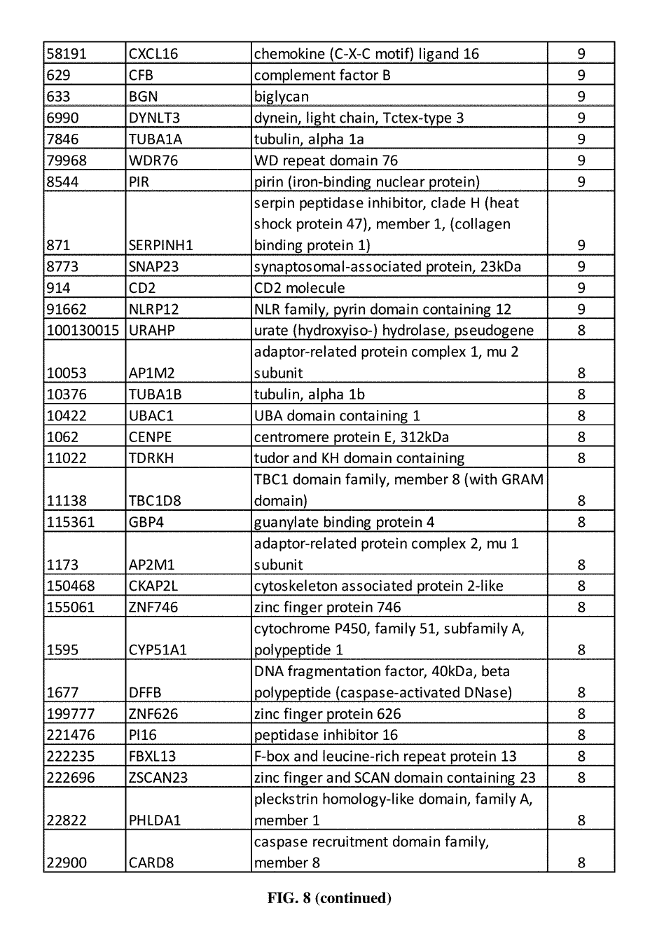

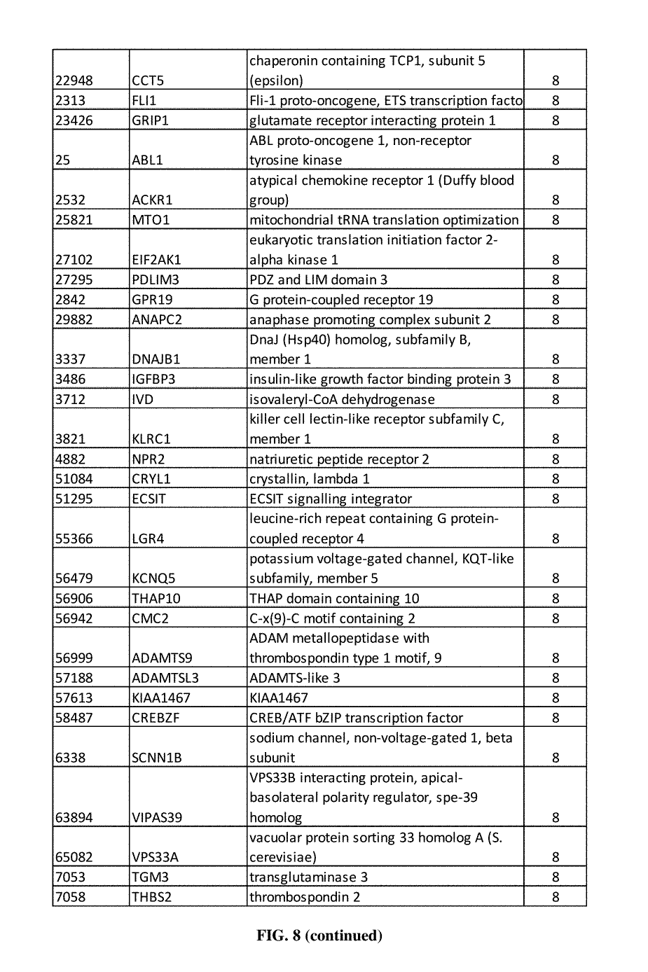

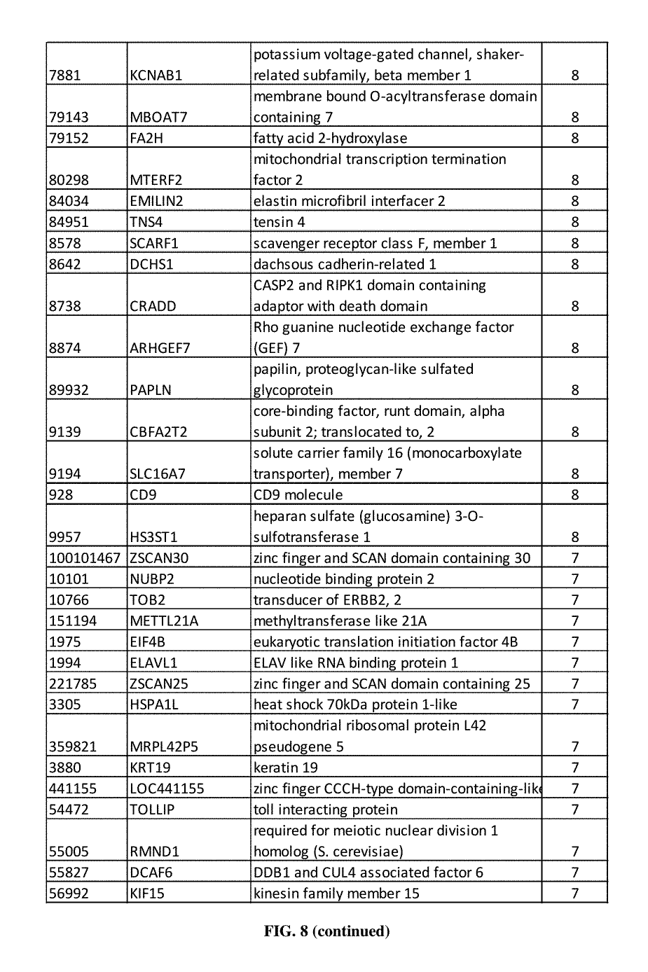

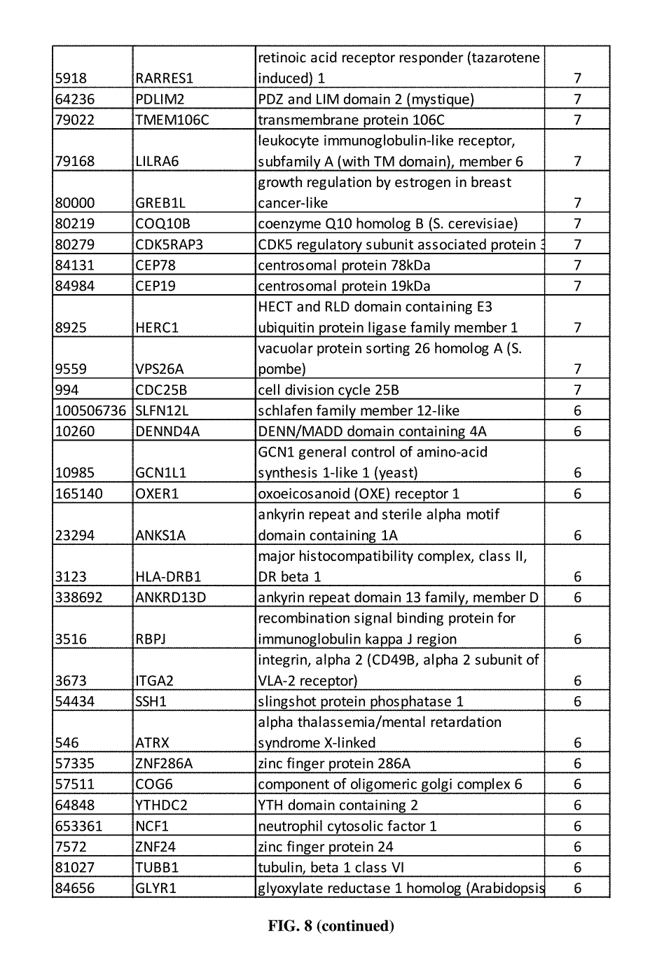

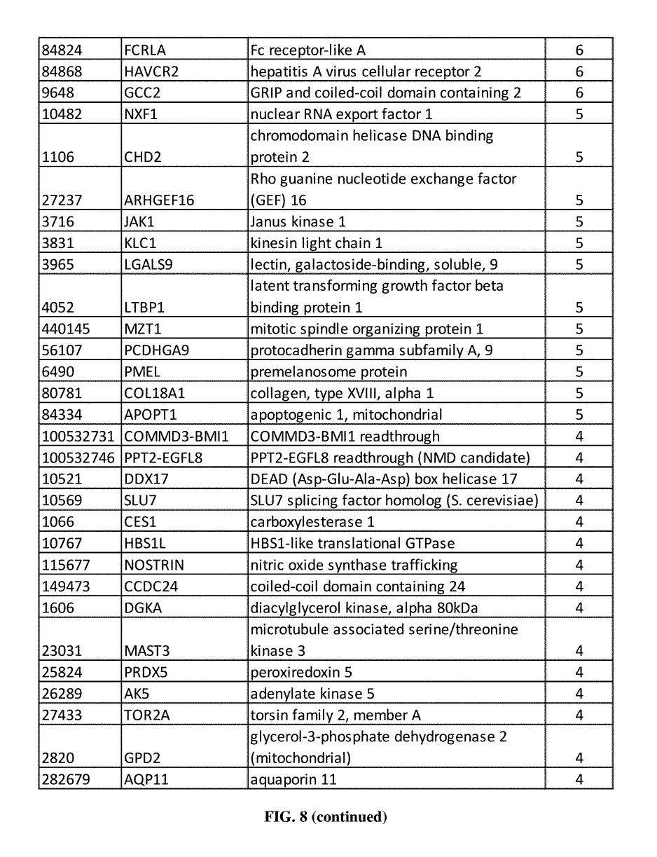

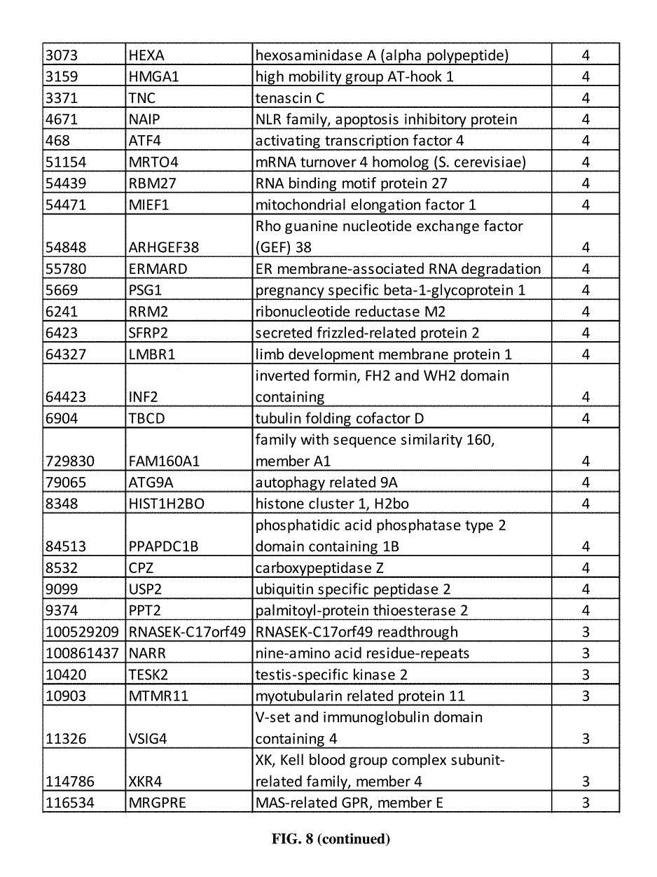

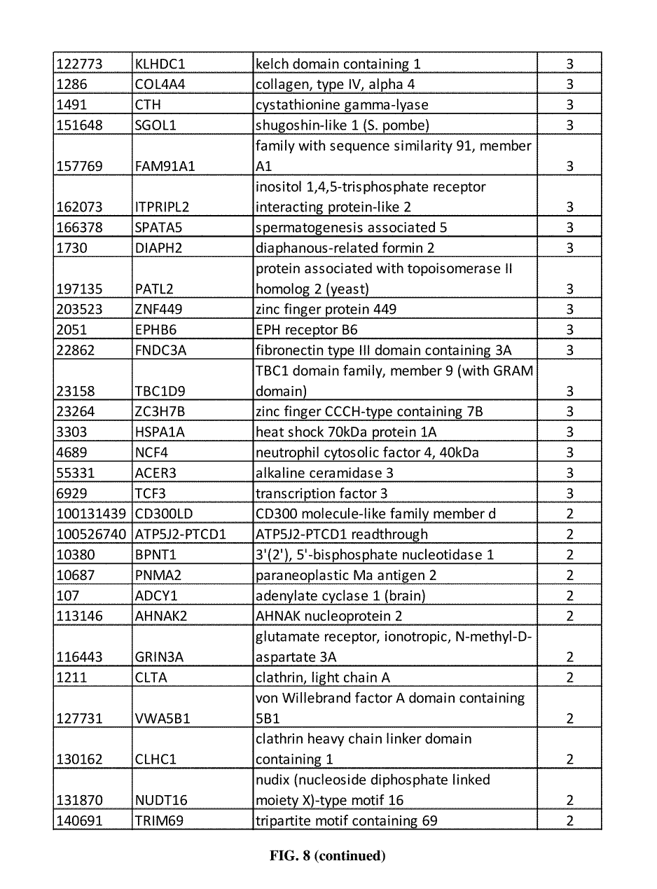

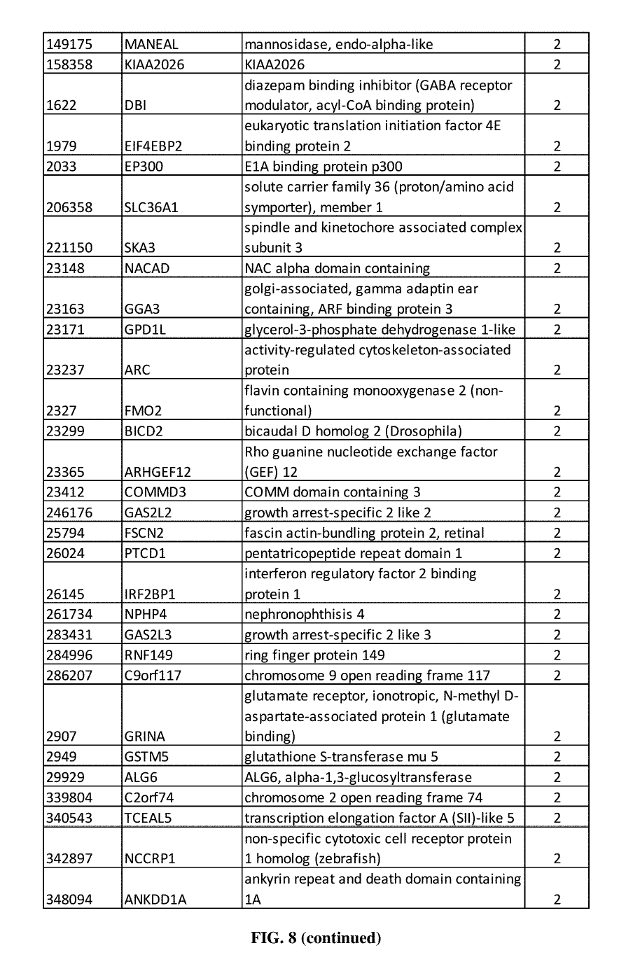

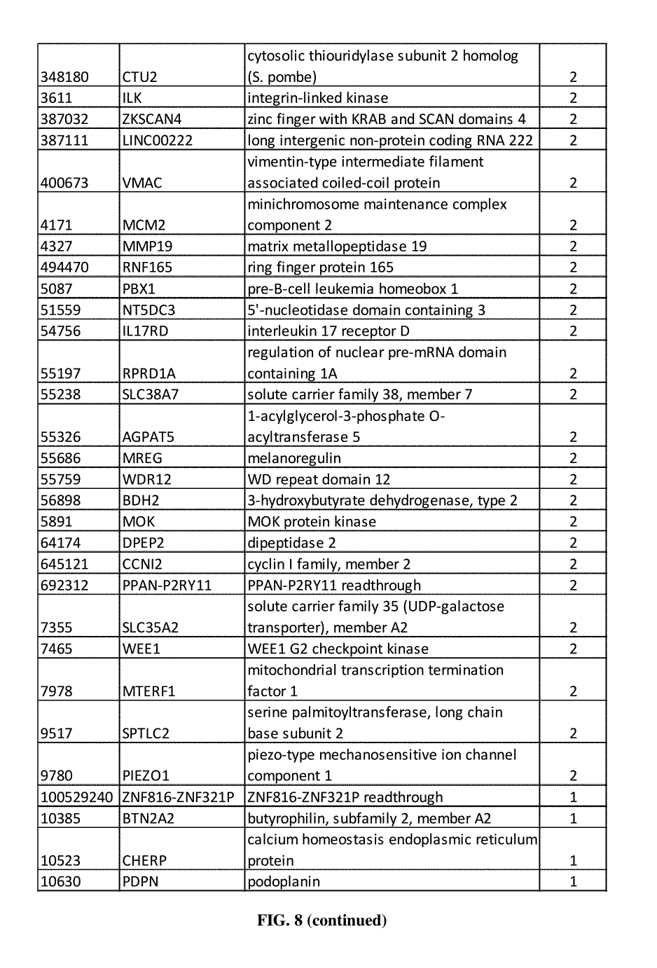

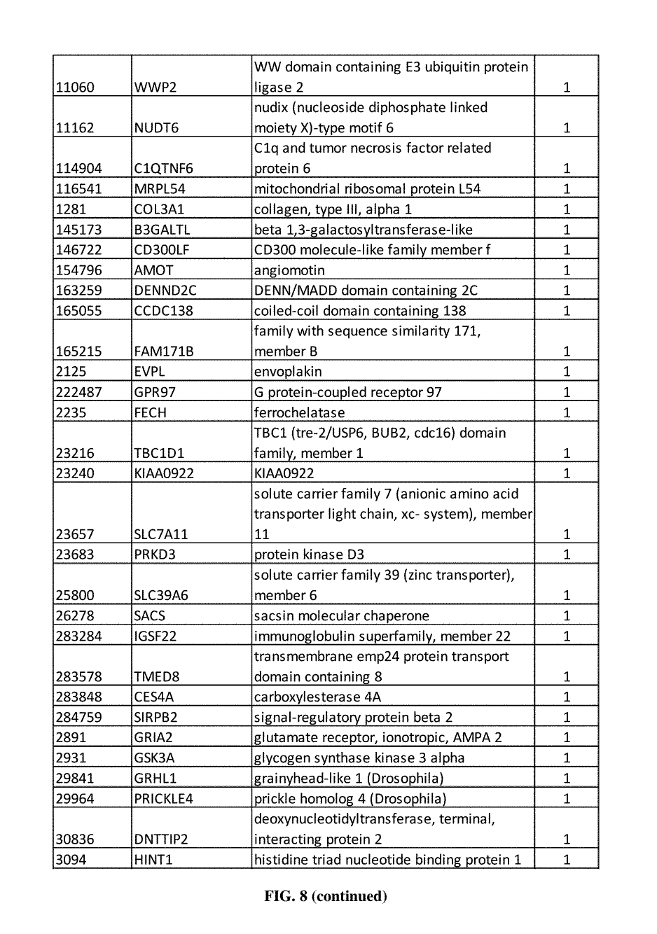

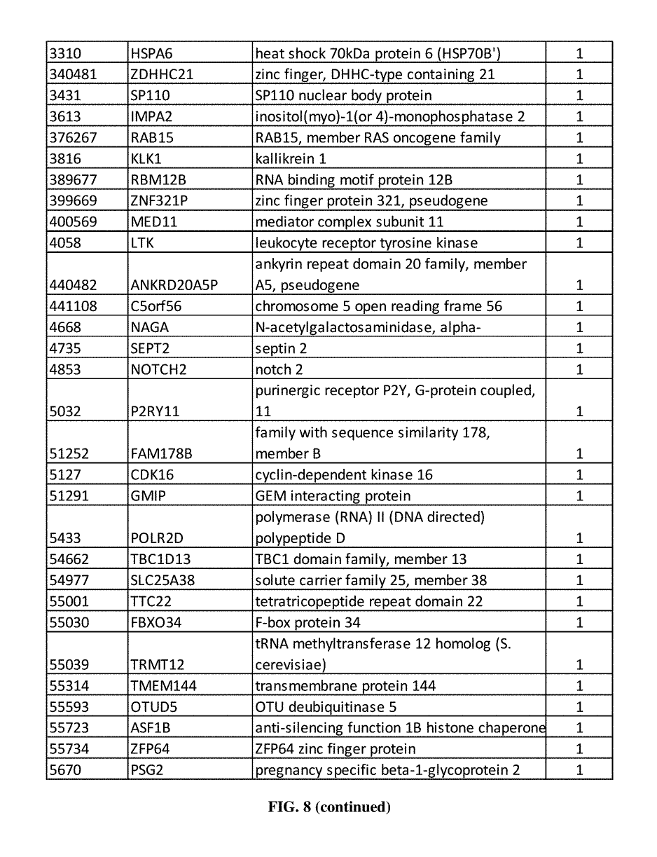

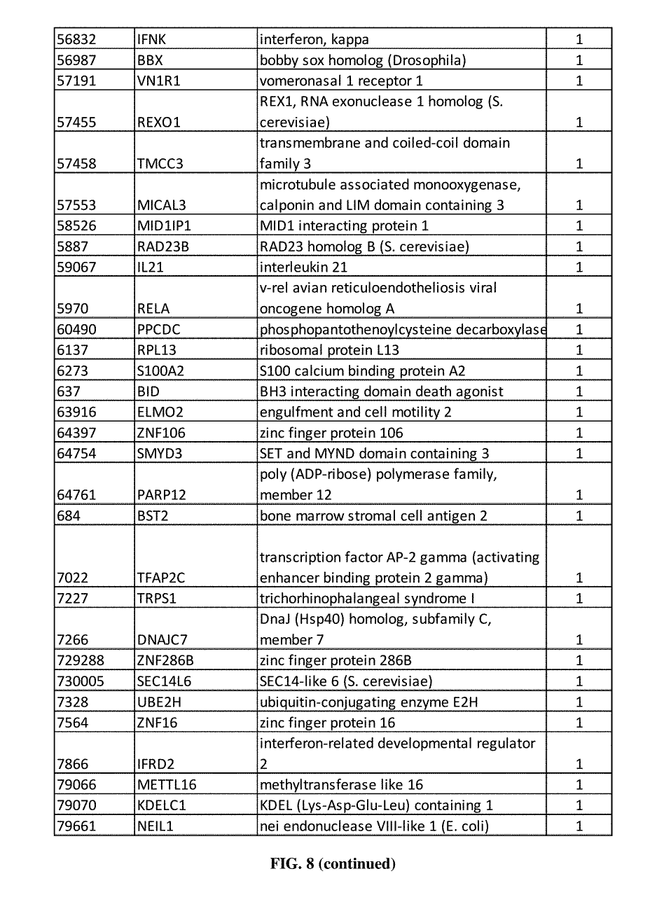

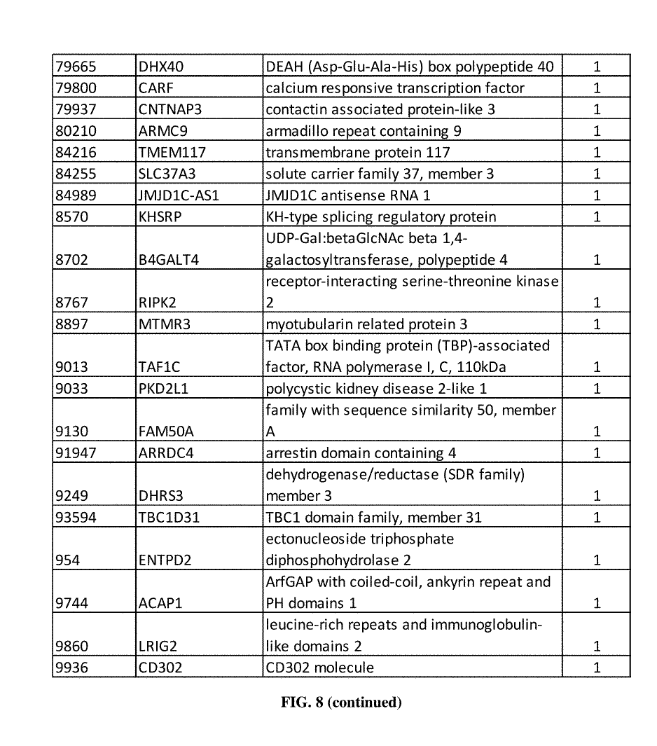

The one or more genes in the first set or second set of genes can include a plurality of genes, such as about 2, 10, 20, 40 genes or more. The one or more genes of the first or second sets can be less than about 10 genes, 20 genes, 50 genes, 60 genes, or about 75 genes. The one or more genes of the first or second sets can be between about 50 and about 400 genes. The first set of genes can comprise genes from FIG. 6 or FIG. 7. The second set of genes can comprise genes from FIG. 8.

The first set and second set of genes can be the same set. For example, the first and second sets of genes may comprise COL1A1, THBS2, or any combination thereof.

The first set and second set of genes can be different sets. The second set of genes may comprise EPHA3, COL1A1, EHF, RAPGEF5, PRICKLE1, TMEM92, ROBO1, C6orf136, SPAG4, GALNT15, LUM, NCAM2, NUP210L, NR2F1, THBS2, PSORS1C1, or any combination thereof. The first set of genes may comprise COL1A1, TMEM92, C1orf87, SPAG4, EHF, COL3A1, GALNT15, NUP210L, PDZRN3, C6orf136, NA, NRXN3, COL6A3, RAPGEF5, PRICKLE1, LUM, ROBO1, BGN, AC019117.2, PRSS3P1, or any combination thereof. The second set of genes may comprise EPHA3, COL1A1, EHF, RAPGEF5, PRICKLE1, TMEM92, ROBO1, C6orf136, SPAG4, GALNT15, LUM, NCAM2, SYNPO2, NUP210L, AMZ1, NR2F1, THBS2, PSORS1C1, FTH1P24, or any combination thereof. The second set of genes may comprise AKAP9, SPRY3, SPRY3, CAMKK2, COL1A1, FITM2, COX6C, VSIG10L, CYC1, KDM1B, MAPK15, ARSG, PAXIP1, DAAM1, AVL9, DMGDH, HLA-DQA1, HLA-DQB1, HLA-DRA, HLA-DRB5, HLA-H, IRF1, MGAT1, P2RX1, PLEK, CCDC93, PPP1R12C, SLC41A3, METTL3, CCAR2, PTPRE, SRL, SLC30A5, BMP4, ZNF133, ICE2, DCAKD, TMX1, TNFSF12, PER2, MCM3AP, or any combination thereof.

### Samples

A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, urine, cerebrospinal fluid, seminal fluid, saliva, sputum, stool, lymph fluid, tissue, or any combination thereof. A sample can be a tissue-specific sample such as a sample obtained from a thyroid tissue, skin, heart, lung, kidney, breast, pancreas, liver, muscle, smooth muscle, bladder, gall bladder, colon, intestine, brain, esophagus, or prostate.

A sample of the present disclosure can be obtained by various methods, such as, for example, fine needle aspiration (FNA), core needle biopsy, vacuum assisted biopsy, incisional biopsy, excisional biopsy, punch biopsy, shave biopsy, skin biopsy, or any combination thereof.

FNA, also referred to as fine needle aspirate biopsy (FNAB), or needle aspirate biopsy (NAB), is a method of obtaining a small amount of tissue from a subject. FNA can be less invasive than a tissue biopsy, which may require surgery and hospitalization of the subject to obtain the tissue biopsy. The needle of a FNA method can be inserted into a tissue mass of a subject to obtain an amount of sample for further analysis. In some cases, two needles can be inserted into the tissue mass. The FNA sample obtained from the tissue mass may be acquired by one or more passages of the needle across the tissue mass. In some cases, the FNA sample can comprise less than about 6×10⁶, 5×10⁶, 4×10⁶, 3×10⁶, 2×10⁶, 1×10⁶ cells or less. The needle can be guided to the tissue mass by ultrasound or other imaging device. The needle can be hollow to permit recovery of the FNA sample through the needle by aspiration or vacuum or other suction techniques.

Samples obtained using methods disclosed herein, such as an FNA sample, may comprise a small sample volume. A sample volume may be less than about 500 microliters (uL), 400 uL, 300 uL, 200 uL, 100 uL, 75 uL, 50 uL, 25 uL, 20 uL, 15 uL, 10 uL, 5 uL, 1 uL, 0.5 uL, 0.1 uL, 0.01 uL or less. The sample volume may be less than about 1 uL. The sample volume may be less than about 5 uL. The sample volume may be less than about 10 uL. The sample volume may be less than about 20 uL. The sample volume may be between about 1 uL and about 10 uL. The sample volume may be between about 10 uL and about 25 uL.

Samples obtained using methods disclosed herein, such as an FNA sample, may comprise small sample weights. The sample weight, such as a tissue weight, may be less than about 100 milligrams (mg), 75 mg, 50 mg, 25 mg, 20 mg, 15 mg, 10 mg, 9 mg, 8 mg, 7 mg, 6 mg, 5 mg, 4 mg, 3 mg, 2 mg, 1 mg, 0.5 mg, 0.1 mg or less. The sample weight may be less than about 20 mg. The sample weight may be less than about 10 mg. The sample weight may be less than about 5 mg. The sample weight may be between about 5 mg and about 20 mg. The sample weight may be between about 1 mg and about 5 ng.

Samples obtained using methods disclosed herein, such as FNA, may comprise small numbers of cells. The number of cells of a single sample may be less than about 10×10⁶ 5.5×10⁶ 5×10⁶ 4.5×10⁶ 4×10⁶ 3.5×10⁶ 3×10⁶ 2.5×10⁶ 2×10⁶ 1.5×10⁶, 1×10⁶, 0.5×10⁶, 0.2×10⁶, 0.1×10⁶ cells or less. The number of cells of a single sample may be less than about 5×10⁶ cells. The number of cells of a single sample may be less than about 4×10⁶ cells. The number of cells of a single sample may be less than about 3×10⁶ cells. The number of cells of a single sample may be less than about 2×10⁶ cells. The number of cells of a single sample may be between about 1×10⁶ and about 5×10⁶ cells. The number of cells of a single sample may be between about 1×10⁶ and about 10×10⁶ cells.

Samples obtained using methods disclosed herein, such as FNA, may comprise small amounts of deoxyribonucleic acid (DNA) or ribonucleic acid (RNA). The amount of DNA or RNA in an individual sample may be less than about 500 nanograms (ng), 400 ng, 300 ng, 200 ng, 100 ng, 75 ng, 50 ng, 45 ng, 40 ng, 35 ng, 30 ng, 25 ng, 20 ng, 15 ng, 10 ng, 5 ng, 1 ng, 0.5 ng, 0.1 ng, or less. The amount of DNA or RNA may be less than about 40 ng. The amount of DNA or RNA may be less than about 25 ng. The amount of DNA or RNA may be less than about 15 ng. The amount of DNA or RNA may be between about 1 ng and about 25 ng. The amount of DNA or RNA may be between about 5 ng and about 50 ng.

RNA yield or RNA amount of a sample can be measured in nanogram to microgram amounts. An example of an apparatus that can be used to measure nucleic acid yield in the laboratory is a NANODROP® spectrophotometer, QUBIT® fluorometer, or QUANTUS™ fluorometer. The accuracy of a NANODROP® measurement may decrease significantly with very low RNA concentration. Quality of data obtained from the methods described herein can be dependent on RNA quantity. Meaningful gene expression or sequence variant data or others can be generated from samples having a low or unmeasurable RNA concentration as measured by NANODROP®. In some cases, gene expression or sequence variant data or others can be generated from a sample having an unmeasurable RNA concentration.

The methods as described herein can be performed using samples with low quantity or quality of polynucleotides, such as DNA or RNA. A sample with low quantity or quality of RNA can be for example a degraded or partially degraded tissue sample. A sample with low quantity or quality of RNA may be a fine needle aspirate (FNA) sample. The RNA quality of a sample can be measured by a calculated RNA Integrity Number (RIN) value. The RIN value is an algorithm for assigning integrity values to RNA measurements. The algorithm can assign a 1 to 10 RIN value, where an RIN value of 10 can be completely intact RNA. A sample as described herein that comprises RNA can have an RIN value of about 9.0, 8.0, 7.0, 6.0, 5.0, 4.0, 3.0, 2.0, 1.0 or less. In some cases, a sample comprising RNA can have an RIN value equal or less than about 8.0. In some cases, a sample comprising RNA can have an RIN value equal or less than about 6.0. In some cases, a sample comprising RNA can have an MN value equal or less than about 4.0. In some cases, a sample can have an RIN value of less than about 2.0.

A sample, such as an FNA sample, may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. A medical professional may obtain a sample from a subject for testing or refer the subject to a testing center or laboratory for the submission of the sample. The medical professional may indicate to the testing center or laboratory the appropriate test or assay to perform on the sample, such as methods of the present disclosure including determining gene sequence data, gene expression levels, sequence variant data, or any combination thereof.

In some cases, a medical professional need not be involved in the initial diagnosis of a disease or the initial sample acquisition. An individual, such as the subject, may alternatively obtain a sample through the use of an over the counter kit. The kit may contain collection unit or device for obtaining the sample as described herein, a storage unit for storing the sample ahead of sample analysis, and instructions for use of the kit.

A sample can be obtained a) pre-operatively, b) post-operatively, c) after a cancer diagnosis, d) during routine screening following remission or cure of disease, e) when a subject is suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

### Cytological Analysis

The methods as described herein, including assessment of risk of occurrence of disease may include cytological analysis of samples. Examples of cytological analysis include cell staining techniques and/or microscope examination performed by any number of methods and suitable reagents including but not limited to: eosin-azure (EA) stains, hematoxylin stains, CYTO-STAIN™, papanicolaou stain, eosin, nissl stain, toluidine blue, silver stain, azocarmine stain, neutral red, or janus green. More than one stain can be used in combination with other stains. In some cases, cells are not stained at all. Cells can be fixed and/or permeabilized with for example methanol, ethanol, glutaraldehyde or formaldehyde prior to or during the staining procedure. In some cases, the cells may not be fixed. Staining procedures can also be utilized to measure the nucleic acid content of a sample, for example with ethidium bromide, hematoxylin, nissl stain or any other nucleic acid stain.

Microscope examination of cells in a sample can include smearing cells onto a slide by standard methods for cytological examination. Liquid based cytology (LBC) methods may be utilized. In some cases, LBC methods provide for an improved approach of cytology slide preparation, more homogenous samples, increased sensitivity and specificity, or improved efficiency of handling of samples, or any combination thereof. In LBC methods, samples can be transferred from the subject to a container or vial containing a LBC preparation solution such as for example CYTYC THINPREP®, SUREPATH™, or MONOPREP® or any other LBC preparation solution. Additionally, the sample may be rinsed from the collection device with LBC preparation solution into the container or vial to ensure substantially quantitative transfer of the sample. The solution containing the sample in LBC preparation solution may then be stored and/or processed by a machine or by one skilled in the art to produce a layer of cells on a glass slide. The sample may further be stained and examined under the microscope in the same way as a conventional cytological preparation.

Samples can be analyzed by immuno-histochemical staining. Immuno-histochemical staining can provide analysis of the presence, location, and distribution of specific molecules or antigens by use of antibodies in a sample (e.g. cells or tissues). Antigens can be small molecules, proteins, peptides, nucleic acids or any other molecule capable of being specifically recognized by an antibody. Samples may be analyzed by immuno-histochemical methods with or without a prior fixing and/or permeabilization step. In some cases, the antigen of interest may be detected by contacting the sample with an antibody specific for the antigen and then nonspecific binding may be removed by one or more washes. The specifically bound antibodies may then be detected by an antibody detection reagent such as for example a labeled secondary antibody, or a labeled avidin/streptavidin. The antigen specific antibody can be labeled directly. Suitable labels for immuno-histochemistry include but are not limited to fluorophores such as fluorescein and rhodamine, enzymes such as alkaline phosphatase and horse radish peroxidase, or radionuclides such as ³²P and ¹²⁵I. Gene product markers that may be detected by immuno-histochemical staining include but are not limited to Her2/Neu, Ras, Rho, EGFR, VEGFR, UbeH10, RET/PTC1, cytokeratin 20, calcitonin, GAL-3, thyroid peroxidase, or thyroglobulin.

Metrics associated with a risk of disease occurrence as disclosed herein, such as gene expression levels of a first gene set or sequence variant data of a second gene set, need not be a characteristic of every cell of a sample found to comprise the risk of disease occurrence. Thus, the methods disclosed herein can be useful for assessing a risk of disease occurrence, such as a cancer, within a tissue where less than all cells within the sample exhibit a complete pattern of the gene expression levels or sequence variant data, or other data indicative of a risk of occurrence of the disease. The gene expression levels, sequence variant data, or others may be either completely present, partially present, or absent within affected cells, as well as unaffected cells of the sample. The gene expression levels, sequence variant data, or others may be present in variable amounts within affected cells. The gene expression levels, sequence variant data, or others may be present in variable amounts within unaffected cells. In some cases, the gene expression levels of a first set of genes or the presence of one or more sequence variants in a second set of genes that correlates with a risk of disease occurrence can be positively detected. In some instances, positive detection can occur in at least 70%, 75%, 80%, 85%, 90%, 95%, or 100% of cells drawn from a sample. In some cases, the gene expression levels of a first set of genes or the presence of one or more sequence variants in a second set of genes can be absent. In some instances, absence of detection can occur in at least 70%, 75%, 80%, 85%, 90%, 95%, or 100% of cells of a corresponding normal, non-disease sample.

Routine cytological or other assays may indicate a sample as negative (without disease), diagnostic (positive diagnosis for disease, such as cancer), ambiguous or suspicious (suggestive of the presence of a disease, such as cancer), or non-diagnostic (providing inadequate information concerning the presence or absence of disease). The methods as described herein may confirm results from the routine cytological assessments or may provide an original assessment similar to a routine cytological assessment in the absence of one. The methods as described herein may classify a sample as malignant or benign, including samples found to be ambiguous or suspicious. The methods may further stratify samples, such as samples known to be malignant, into low risk and medium-to-high risk groups of disease occurrence, including samples found to be ambiguous or suspicious.

### Diseases

A disease, as disclosed herein, can include thyroid cancer. Thyroid cancer can include any subtype of thyroid cancer, including but not limited to, any malignancy of the thyroid gland such as papillary thyroid cancer (PTC), follicular thyroid cancer (FTC), follicular variant of papillary thyroid carcinoma (FVPTC), medullary thyroid carcinoma (MTC), follicular carcinoma (FC), Hurthle cell carcinoma (TIC), and/or anaplastic thyroid cancer (ATC). In some cases, the thyroid cancer can be differentiated. In some cases, the thyroid cancer can be undifferentiated.

A thyroid tissue sample can be classified using the methods of the present disclosure as comprising one or more benign or malignant tissue types (e.g. a cancer subtype), including but not limited to follicular adenoma (FA), nodular hyperplasia (NHP), lymphocytic thyroiditis (LCT), and Hurthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), and anaplastic thyroid carcinoma (ATC), renal carcinoma (RCC), breast carcinoma (BCA), melanoma (MMN), B cell lymphoma (BCL), or parathyroid (PTA).

Other types of cancer of the present disclosure can include but are not limited to adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinemia.

A disease, as disclosed herein, can include hyperproliferative disorders. Malignant hyperproliferative disorders can be stratified into risk groups, such as a low risk group and a medium-to-high risk group. Hyperproliferative disorders can include but are not limited to cancers, hyperplasias, or neoplasias. In some cases, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotrophic virus-type 1 (HTLV-1) and adult T-cell leukemia/lymphoma; and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Müllerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some cases, the diseases stratified, classified, characterized, or diagnosed by the methods of the present disclosure include but are not limited to thyroid disorders such as for example benign thyroid disorders including but not limited to follicular adenomas, Hurthle cell adenomas, lymphocytic thyroiditis, and thyroid hyperplasia. In some cases, the diseases stratified, classified, characterized, or diagnosed by the methods of the present disclosure include but are not limited to malignant thyroid disorders such as for example follicular carcinomas, follicular variant of papillary thyroid carcinomas, medullary carcinomas, and papillary carcinomas.

Diseases of the present disclosure can include a genetic disorder. A genetic disorder is an illness caused by abnormalities in genes or chromosomes. Genetic disorders can be grouped into two categories: single gene disorders and multifactorial and polygenic (complex) disorders. A single gene disorder can be the result of a single mutated gene. Inheriting a single gene disorder can include but not be limited to autosomal dominant, autosomal recessive, X-linked dominant, X-linked recessive, Y-linked and mitochondrial inheritance. Only one mutated copy of the gene can be necessary for a person to be affected by an autosomal dominant disorder. Examples of autosomal dominant type of disorder can include but are not limited to Huntington's disease, Neurofibromatosis 1, Marfan Syndrome, Hereditary nonpolyposis colorectal cancer, or Hereditary multiple exostoses. In autosomal recessive disorders, two copies of the gene must be mutated for a subject to be affected by an autosomal recessive disorder. Examples of this type of disorder can include but are not limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets. Some X-linked dominant conditions such as Rett syndrome, Incontinentia Pigmenti type 2 and Aicardi Syndrome can be fatal. X-linked recessive disorders are also caused by mutations in genes on the X chromosome. Examples of this type of disorder can include but are not limited to Hemophilia A, Duchenne muscular dystrophy, red-green color blindness, muscular dystrophy and Androgenetic alopecia. Y-linked disorders are caused by mutations on the Y chromosome. Examples can include but are not limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

Genetic disorders may also be complex, multifactorial or polygenic. Polygenic genetic disorders can be associated with the effects of multiple genes in combination with lifestyle and environmental factors. Although complex genetic disorders can cluster in families, they do not have a clear-cut pattern of inheritance. Multifactorial or polygenic disorders can include heart disease, diabetes, asthma, autism, autoimmune diseases such as multiple sclerosis, cancers, ciliopathies, cleft palate, hypertension, inflammatory bowel disease, mental retardation or obesity.

Other genetic disorders can include but are not limited to 1p36 deletion syndrome, 21-hydroxylase deficiency, 22q11.2 deletion syndrome, aceruloplasminemia, achondrogenesis, type II, achondroplasia, acute intermittent porphyria, adenylosuccinate lyase deficiency, Adrenoleukodystrophy, Alexander disease, alkaptonuria, alpha-1 antitrypsin deficiency, Alstrom syndrome, Alzheimer's disease (type 1, 2, 3, and 4), Amelogenesis Imperfecta, amyotrophic lateral sclerosis, Amyotrophic lateral sclerosis type 2, Amyotrophic lateral sclerosis type 4, amyotrophic lateral sclerosis type 4, androgen insensitivity syndrome, Anemia, Angelman syndrome, Apert syndrome, ataxia-telangiectasia, Beare-Stevenson cutis gyrata syndrome, Benjamin syndrome, beta thalassemia, biotinidase deficiency, Birt-Hogg-Dube syndrome, bladder cancer, Bloom syndrome, Bone diseases, breast cancer, Camptomelic dysplasia, Canavan disease, Cancer, Celiac Disease, Chronic Granulomatous Disorder (CGD), Charcot-Marie-Tooth disease, Charcot-Marie-Tooth disease Type 1, Charcot-Marie-Tooth disease Type 4, Charcot-Marie-Tooth disease Type 2, Charcot-Marie-Tooth disease Type 4, Cockayne syndrome, Coffin-Lowry syndrome, collagenopathy types II and XI, Colorectal Cancer, Congenital absence of the vas deferens, congenital bilateral absence of vas deferens, congenital diabetes, congenital erythropoietic porphyria, Congenital heart disease, congenital hypothyroidism, Connective tissue disease, Cowden syndrome, Cri du chat syndrome, Crohn's disease, fibrostenosing, Crouzon syndrome, Crouzonodermoskeletal syndrome, cystic fibrosis, De Grouchy Syndrome, Degenerative nerve diseases, Dent's disease, developmental disabilities, DiGeorge syndrome, Distal spinal muscular atrophy type V, Down syndrome, Dwarfism, Ehlers-Danlos syndrome, Ehlers-Danlos syndrome arthrochalasia type, Ehlers-Danlos syndrome classical type, Ehlers-Danlos syndrome dermatosparaxis type, Ehlers-Danlos syndrome kyphoscoliosis type, vascular type, erythropoietic protoporphyria, Fabry's disease, Facial injuries and disorders, factor V Leiden thrombophilia, familial adenomatous polyposis, familial dysautonomia, fanconi anemia, FG syndrome, fragile X syndrome, Friedreich ataxia, Friedreich's ataxia, G6PD deficiency, galactosemia, Gaucher's disease (type 1, 2, and 3), Genetic brain disorders, Glycine encephalopathy, Haemochromatosis type 2, Haemochromatosis type 4, Harlequin Ichthyosis, Head and brain malformations, Hearing disorders and deafness, Hearing problems in children, hemochromatosis (neonatal, type 2 and type 3), hemophilia, hepatoerythropoietic porphyria, hereditary coproporphyria, Hereditary Multiple Exostoses, hereditary neuropathy with liability to pressure palsies, hereditary nonpolyposis colorectal cancer, homocystinuria, Huntington's disease, Hutchinson Gilford Progeria Syndrome, hyperoxaluria, primary, hyperphenylalaninemia, hypochondrogenesis, hypochondroplasia, idic15, incontinentia pigmenti, Infantile Gaucher disease, infantile-onset ascending hereditary spastic paralysis, Infertility, Jackson-Weiss syndrome, Joubert syndrome, Juvenile Primary Lateral Sclerosis, Kennedy disease, Klinefelter syndrome, Kniest dysplasia, Krabbe disease, Learning disability, Lesch-Nyhan syndrome, Leukodystrophies, Li-Fraumeni syndrome, lipoprotein lipase deficiency, familial, Male genital disorders, Marfan syndrome, McCune-Albright syndrome, McLeod syndrome, Mediterranean fever, familial, Menkes disease, Menkes syndrome, Metabolic disorders, methemoglobinemia beta-globin type, Methemoglobinemia congenital methaemoglobinaemia, methylmalonic acidemia, Micro syndrome, Microcephaly, Movement disorders, Mowat-Wilson syndrome, Mucopolysaccharidosis (MPS I), Muenke syndrome, Muscular dystrophy, Muscular dystrophy, Duchenne and Becker type, muscular dystrophy, Duchenne and Becker types, myotonic dystrophy, Myotonic dystrophy type 1 and type 2, Neonatal hemochromatosis, neurofibromatosis, neurofibromatosis 1, neurofibromatosis 2, Neurofibromatosis type I, neurofibromatosis type II, Neurologic diseases, Neuromuscular disorders, Niemann-Pick disease, Nonketotic hyperglycinemia, nonsyndromic deafness, Nonsyndromic deafness autosomal recessive, Noonan syndrome, osteogenesis imperfecta (type I and type III), otospondylomegaepiphyseal dysplasia, pantothenate kinase-associated neurodegeneration, Patau Syndrome (Trisomy 13), Pendred syndrome, Peutz-Jeghers syndrome, Pfeiffer syndrome, phenylketonuria, porphyria, porphyria cutanea tarda, Prader-Willi syndrome, primary pulmonary hypertension, prion disease, Progeria, propionic acidemia, protein C deficiency, protein S deficiency, pseudo-Gaucher disease, pseudoxanthoma elasticum, Retinal disorders, retinoblastoma, retinoblastoma FA Friedreich ataxia, Rett syndrome, Rubinstein-Taybi syndrome, Sandhoff disease, sensory and autonomic neuropathy type III, sickle cell anemia, skeletal muscle regeneration, Skin pigmentation disorders, Smith Lemli Opitz Syndrome, Speech and communication disorders, spinal muscular atrophy, spinal-bulbar muscular atrophy, spinocerebellar ataxia, spondyloepimetaphyseal dysplasia, Strudwick type, spondyloepiphyseal dysplasia congenita, Stickler syndrome, Stickler syndrome COL2A1, Tay-Sachs disease, tetrahydrobiopterin deficiency, thanatophoric dysplasia, thiamine-responsive megaloblastic anemia with diabetes mellitus and sensorineural deafness, Thyroid disease, Tourette's Syndrome, Treacher Collins syndrome, triple X syndrome, tuberous sclerosis, Turner syndrome, Usher syndrome, variegate porphyria, von Hippel-Lindau disease, Waardenburg syndrome, Weissenbacher-Zweymüller syndrome, Wilson disease, Wolf-Hirschhorn syndrome, Xeroderma Pigmentosum, X-linked severe combined immunodeficiency, X-linked sideroblastic anemia, or X-linked spinal-bulbar muscle atrophy.

### Stratifying Risk of Occurrence or Recurrence

A risk of occurrence of disease can be stratifying samples into risk subgroups. Subgroups can comprise samples with a low risk of probability of disease occurrence and samples with a medium-to-high risk of probability of disease occurrence. Subgroups can comprise low risk, medium risk, and high risk groups. Low risk can comprise samples with about a 1%, 5%, 10%, 15%, 20%, 25%, 30%, 35%, 40%, or about 45% risk of probability of disease occurrence. Low risk can comprise samples with between about a 1% and about a 25% risk probability of disease occurrence. Low risk can comprise samples with between about a 1% and about a 30% risk of probability of disease occurrence. Low risk can comprise samples with between about a 1% and about a 40% risk of probability of disease occurrence. Medium-to-high risk can comprise samples with about a 55%, 60%, 65%, 70%, 75%, 80%, 85% 90%, 95%, or 100% risk of probability of disease occurrence. Medium-to-high risk can comprise samples with between about a 50% and about a 100% risk of probability of disease occurrence. Medium-to-high risk can comprise samples with between about a 55% and about a 100% risk of probability of disease occurrence. Medium-to-high risk can comprise samples between about a 60% and about a 100% risk of probability of disease occurrence.

A sample can be stratified into a low risk or a medium-to-high risk group with an accuracy of at least 50%, 60%, 70%, 75%, 80%, 85%, 90%, 95%, 96%, 97%, 98%, 99% or more, including samples identified as cytologically ambiguous or suspicious or indeterminate. A sample can be stratified with an accuracy of at least 70%. A sample can be stratified with an accuracy of at least 80%. A sample can be stratified with an accuracy of at least 90%. A sample can be identified as benign, malignant, or non-diagnostic with an accuracy of greater than 50%, 60%, 70%, 75%, 80%, 85%, 90% 95%, 96%, 97%, 98%, 99% or more, including samples identified as cytologically ambiguous or suspicious or indeterminate. Accuracy can be calculated using a classifier.

A sample can be stratified into a low risk or a medium-to-high risk group with a specificity of at least 50%, 60%, 70%, 75%, 80%, 85%, 90%, 95% 96%, 97%, 98%, 99% or more, including samples identified as cytologically ambiguous or suspicious or indeterminate. A sample can be stratified with an accuracy of at least 70%. A sample can be stratified with an accuracy of at least 80%. A sample can be stratified with an accuracy of at least 90%. A sample can be identified as benign, malignant, or non-diagnostic with a specificity of greater than 50%, 60%, 70%, 75%, 80% 85%, 90%, 95%, 96%, 97%, 98%, 99% or more, including samples identified as cytologically ambiguous or suspicious or indeterminate. Specificity can be calculated using a classifier.

Methods as described herein for stratifying risk of occurrence of a disease, classifying samples as benign, malignant, or non-diagnostic can have a positive predictive value of at least 95%, 95.5% 96%, 96.5% 97%, 97.5%, 98%, 98.5%, 99%, 99.5% or more; and/or a negative predictive value of at least 95%, 95.5%, 96%, 96.5%, 97%, 97.5%, 98%, 98.5% 99%, 99.5% or more. Positive predictive value (PPV), or precision rate, or post-test probability of disease, can be the proportion of subjects with positive test results who are correctly diagnosed or correctly stratified into risk groups. It can be an important measure because it can reflect the probability that a positive test reflects the underlying disease being tested for. Its value can depend on the prevalence of the disease, which may vary. The negative predictive value (NPV) can be the proportion of subjects with negative test results who are correctly diagnosed. PPV and NPV measurements can be derived using appropriate disease subtype prevalence estimates. For subtype specific estimates, disease prevalence may sometimes be incalculable because there may not be any available samples.

A sample can be classified into one or more of the following: benign (free of disease), malignant (positive diagnosis for a disease), or non-diagnostic (providing inadequate information concerning the presence or absence of a disease). A sample found to be malignant can be stratified into a risk of disease occurrence such as a low risk of disease occurrence or medium-to-high risk of disease occurrence. Samples can be classified into benign versus suspicious (suspected to be positive for a disease) categories. Samples can be further classified for a disease subtype such as by identifying the presence or absence of one or more cancer subtypes. A certain molecular pathway may be indicated to be involved in the disease, or a certain grade or stage of a particular disease (such as I, II, III, or IV cancer) can also be indicated. In some cases, the stratified risk of occurrence may inform an appropriate therapeutic intervention, such as a specific drug regimen, or a surgical intervention like a thyroidectomy or a hemi-thyroidectomy.

The classifier or trained algorithm of the present disclose can be used to stratify a sample into low or medium-to-high risk groups and/or to classify a sample as benign, malignant, suspicious or non-diagnostic, or others. One or more selected feature spaces such as gene expression level and sequence variant data can be provided alone or in combination to a classifier or trained algorithm. Illustrative algorithms can include but are not limited to methods that reduce the number of variables such as a principal component analysis algorithm, partial least squares method, or independent component analysis algorithm. Illustrative algorithms can include methods that handle large numbers of variables directly such as statistical methods or methods based on machine learning techniques. Statistical methods can include penalized logistic regression, prediction analysis of microarrays (PAM), methods based on shrunken centroids, support vector machine analysis, or regularized linear discriminant analysis. Machine learning techniques can include bagging procedures, boosting procedures, random forest algorithms, or any combination thereof.

The classifier or trained algorithm of the present disclosure can comprise two or more feature spaces. The two or more feature spaces can be unique or distinct from one another. Individual feature spaces can comprise types of information about a sample, such as gene expression level data or sequence variant data. Combining two or more feature spaces in a classifier can produce a higher level of accuracy of the risk stratifying or classifying than producing risk stratification using a single feature space. The dynamic ranges of the individual feature spaces can be different, such as at least 1 or 2 orders of magnitude different. For example, the dynamic range of the gene expression level feature space may be between 0 and about 300 and the dynamic range of sequence variant feature space may be between 0 and about 20.

Individual feature spaces can comprise a set of genes, such as a first set of genes of the first feature space and a second set of genes of the second feature space. A set of genes of an individual feature space can be associated with a risk of occurrence of risk. The first set of genes and the second set of genes can be the same set. The first set of genes and the second set of genes can be different sets. The first set of genes or the second set of genes can comprise less than about 1000, 500, 400, 300, 200, 100, 75, 70, 65, 60, 55, 50, 45, 40, 35, 30, 25, 20, 15, 10, 5 genes or less. The first set of genes or the second set of genes can comprise less than about 10 genes. The first set of genes or the second set of genes can comprise less than about 50 genes. The first set of genes or the second set of genes can comprise less than about 75 genes. The first set of genes or the second set of genes can comprise between about 50 and about 400 genes. The first set of genes or the second set of genes can comprise between about 50 and about 200 genes. The first set of genes or the second set of genes can comprise between about 10 and about 600 genes.

The first set of genes can comprise genes listed in FIG. 6. The first set of genes can comprise genes listed in FIG. 7. The first set of genes can comprise COL1A1, THBS2, or any combination thereof. The first set of genes can comprise COL1A1, TMEM92, C1orf87, SPAG4, EHF, COL3A1, GALNT15, NUP210L, PDZRN3, C6orf136, NA, NRXN3, COL6A3, RAPGEF5, PRICKLE1, LUM, ROBO1, BGN, AC019117.2, PRSS3P1, or any combination thereof.

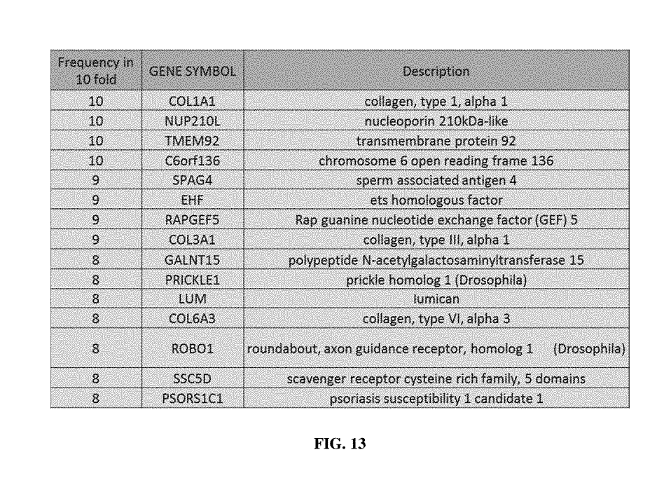

The first set of genes can comprise genes listed in FIG. 13. The first set of genes can comprise COL1A1, NUP210L, TMEM92, C6orf136, SPAG4, EHF, RAPGEF5, COL3A1, GALNT15, PRICKLE1, LUM, COL6A3, ROBO1, SSC5D, PSORS1C1, or any combination thereof. The first set of genes can be selected from the group consisting of COL1A1, NUP210L, TMEM92, C6orf136, SPAG4, EHF, RAPGEF5, COL3A1, GALNT15, PRICKLE1, COL6A3, ROBO1, SSC5D, PSORS1C1, and any combination thereof. The first set of genes can comprise COL1A1. The first set of genes can comprise NUP210L. The first set of genes can comprise TMEM92. The first set of genes can comprise C6orf136. The first set of genes can comprise SPAG4. The first set of genes can comprise EHF. The first set of genes can comprise RAPGEF5. The first set of genes can comprise COL3A1, The first set of genes can comprise GALNT15. The first set of genes can comprise PRICKLE1. The first set of genes can comprise LUM. The first set of genes can comprise COL6A3. The first set of genes can comprise ROBO1. The first set of genes can comprise SSC5D. The first set of genes can comprise PSORS1C1.

The second set of genes can comprise those genes listed in FIG. 8. The second set of genes can comprise COL1A1, THBS2, or any combination thereof. The second set of genes can comprise EPHA3, COL1A1, EHF, RAPGEF5, PRICKLE1, TMEM92, ROBO1, C6orf136, SPAG4, GALNT15, LUM, NCAM2, NUP210L, NR2F1, THBS2, PSORS1C1, or any combination thereof. The second set of genes can comprise EPHA3, COL1A1, EHF, RAPGEF5, PRICKLE1, TMEM92, ROBO1, C6orf136, SPAG4, GALNT15, LUM, NCAM2, SYNPO2, NUP210L, AMZ1, NR2F1, THBS2, PSORS1C1, FTH1P24, or any combination thereof. The second set of genes can comprise AKAP9, SPRY3, SPRY3, CAMKK2, COL1A1, FITM2, COX6C, VSIG10L, CYC1, KDM1B, MAPK15, ARSG, PAXIP1, DAAM1, AVL9, DMGDH, HLA-DQA1, HLA-DQB1, HLA-DRA, HLA-DRB5, HLA-H, IRF1, MGAT1, P2RX1, PLEK, CCDC93, PPP1R12C, SLC41A3, METTL3, CCAR2, PTPRE, SRL, SLC30A5, BMP4, ZNF133, ICE2, DCAKD, TMX1, TNFSF12, PER2, MCM3AP, or any combination thereof.

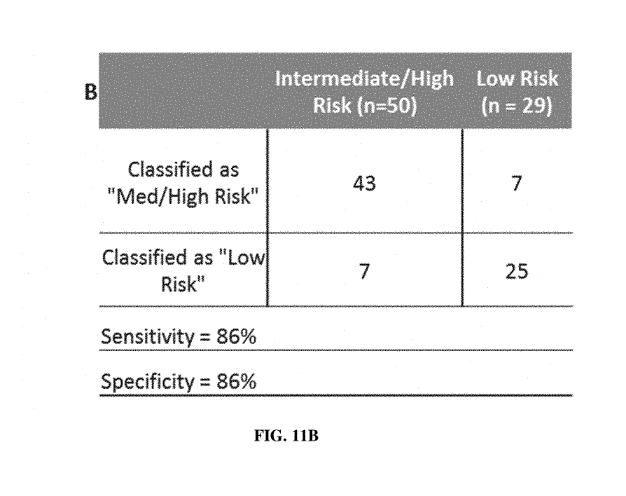

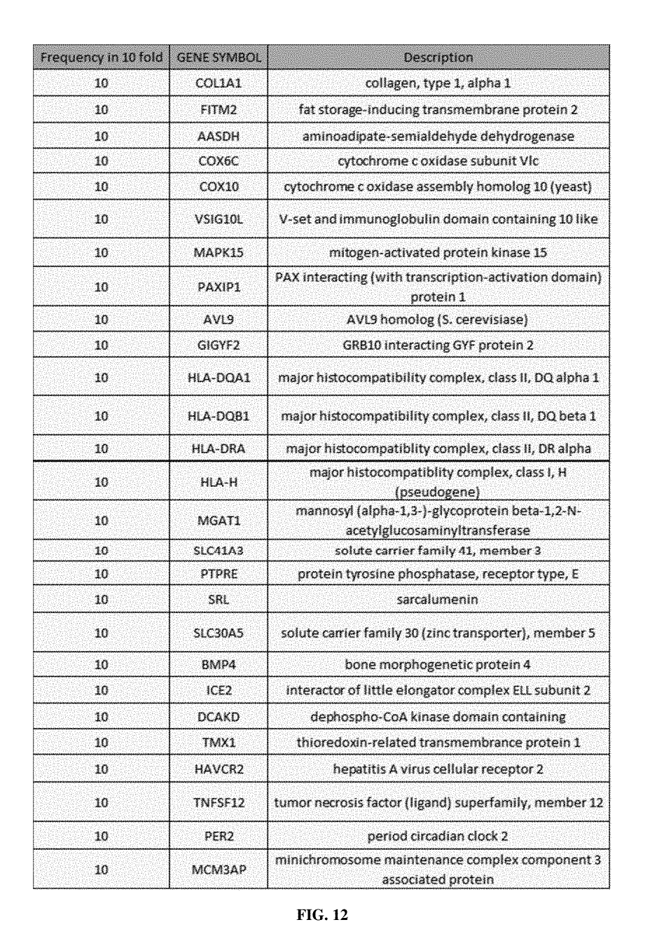

The second set of genes can comprise genes listed in FIG. 12. The second set of genes can comprise COL1A1, FITM2, AASDH, COX6C, COX10, VSIG10L, MAPK15, PAXIP1, AVL9, GIGYF2, HLA-DQA1, HLA-DQB1, HLA-DRA, HLA-H, MGAT1, SLC41A3, PTPRE, SRL, SLC30A5, BMP4, ICE2, DCAKD, TMX1, HAVCR2, TNFSF12, PER2, MCM3AP, or any combination thereof. The second set of genes can be selected from the group consisting of COL1A1, FITM2, AASDH, COX6C, COX10, VSIG10L, MAPK15, PAXIP1, AVL9, GIGYF2, HLA-DQA1, HLA-DQB1, HLA-DRA, HLA-H, MGAT1, SLC41A3, PTPRE, SRL, SLC30A5, BMP4, ICE2, DCAKD, TMX1, HAVCR2, TNFSF12, PER2, MCM3AP, and any combination thereof. The second set of genes can comprise COL1A1. The second set of genes can comprise FITM2. The second set of genes can comprise AASDH. The second set of genes can comprise COX6C. The second set of genes can comprise COX10. The second set of genes can comprise VSIG10L. The second set of genes can comprise MAPK15. The second set of genes can comprise PAXIP1. The second set of genes can comprise AVL9. The second set of genes can comprise GIGYF2. The second set of genes can comprise HLA-DQA1. The second set of genes can comprise HLA-DQB1. The second set of genes can comprise HLA-DRA. The second set of genes can comprise HLA-H. The second set of genes can comprise MGAT1. The second set of genes can comprise SLC41A3. The second set of genes can comprise PTPRE. The second set of genes can comprise SRL. The second set of genes can comprise SLC30A5. The second set of genes can comprise BMP4. The second set of genes can comprise ICE2. The second set of genes can comprise DCAKD. The second set of genes can comprise TMX1. The second set of genes can comprise HAVCR2. The second set of genes can comprise TNFSF12. The second set of genes can comprise PER2. The second set of genes can comprise MCM3AP.

The classifier or trained algorithm of the present disclosure can be trained using a set of samples, such as a sample cohort. The sample cohort can comprise about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise about 100 independent samples. The sample cohort can comprise about 200 independent samples. The sample cohort can comprise between about 100 and about 500 independent samples. The independent samples can be from subjects having been diagnosed with a disease, such as cancer, from healthy subjects, or any combination thereof.

The sample cohort can comprise samples from about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from about 100 different individuals. The sample cohort can comprise samples from about 200 different individuals. The different individuals can be individuals having been diagnosed with a disease, such as cancer, health individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations include, hut are not limited to, test centers, medical facilities, medical offices, post office addresses, cities, counties, states, nations, or continents. In some cases, a classifier that is trained using sample cohorts from the United States may need to be re-trained for use on sample cohorts from other geographical regions (e.g., India, Asia, Europe, Africa, etc.).

A classifier or trained algorithm may produce a unique output each time it is run. For example, using different samples with the same classifier can produce a unique output each time the classifier is run. Using the same samples with the same classifier can produce a unique output each time the classifier is run. Using the same samples to train a classifier more than one time, may result in unique outputs each time the classifier is run.

Characteristics of a sample can be compared to characteristics of a reference set. The comparing can be performed by the classifier. More than one characteristic of a sample can be combined to formulate a risk of disease occurrence. The combining can be performed by the classifier. For example, sequences obtained from a sample can be compared to a reference set to determine the presence of one or more sequence variants in a sample. In some cases, gene expression levels of one or more genes from a sample can be compared to expression levels of a reference set of genes to determine the presence of differential gene expression of one or more genes. The reference set can comprise one or more housekeeping genes. The reference set can comprise known sequence variants or expression levels of genes known to be associated with a particular disease or known to be associated with a non-disease state. The classifier or trained algorithm can perform the comparing, combining, statistical evaluation, or further analysis of results, or any combination thereof. Separate reference sets may be provided for different feature spaces. For example, sequence variant data may be compared to a sequence variant data reference set. A gene expression level data may be compared to a gene expression level reference set. In some cases, multiple feature spaces may be compared to the same reference set.

In some cases, sequence variants of a particular gene may or may not affect the gene expression level of that same gene. A sequence variant of a particular gene may affect the gene expression level of one or more different genes that may be located adjacent to and distal from the particular gene with the sequence variant. The presence of one or more sequence variants can have downstream effects on one or more genes. A sequence variant of a particular gene may perturb one or more signaling pathways, may cause ribonucleic acid (RNA) transcriptional regulation changes, may cause amplification of deoxyribonucleic acid (DNA), may cause multiple transcript copies to be produced, may cause excessive protein to be produced, may cause single base pairs, multi-base pairs, partial genes or one or more genes to be removed from the sequence.

Data from the methods described, such as gene expression levels or sequence variant data can be further analyzed using feature selection techniques such as filters which can assess the relevance of specific features by looking at the intrinsic properties of the data, wrappers which embed the model hypothesis within a feature subset search, or embedded protocols in which the search for an optimal set of features is built into a classifier algorithm.

Filters useful in the methods of the present disclosure can include (1) parametric methods such as the use of two sample t-tests, analysis of variance (ANOVA) analyses, Bayesian frameworks, or Gamma distribution models (2) model free methods such as the use of Wilcoxon rank sum tests, between-within class sum of squares tests, rank products methods, random permutation methods, or threshold number of misclassification (TNoM) which involves setting a threshold point for fold-change differences in expression between two datasets and then detecting the threshold point in each gene that minimizes the number of mis-classifications or (3) multivariate methods such as bivariate methods, correlation based feature selection methods (CFS), minimum redundancy maximum relevance methods (MRMR), Markov blanket filter methods, and uncorrelated shrunken centroid methods. Wrappers useful in the methods of the present disclosure can include sequential search methods, genetic algorithms, or estimation of distribution algorithms. Embedded protocols can include random forest algorithms, weight vector of support vector machine algorithms, or weights of logistic regression algorithms.

Statistical evaluation of the results obtained from the methods described herein can provide a quantitative value or values indicative of one or more of the following: the likelihood of risk assessment accuracy; the likelihood of diagnostic accuracy; the likelihood of disease, such as cancer; the likelihood of a particular disease, such as a tissue-specific cancer, for example, thyroid cancer; and the likelihood of the success of a particular therapeutic intervention. Thus a medical professional, who may not be trained in genetics or molecular biology, need not understand gene expression level or sequence variant data results. Rather, data can be presented directly to the medical professional in its most useful form to guide care or treatment of the subject. Statistical evaluation, combination of separate data results, and reporting useful results can be performed by a classifier or trained algorithm. Statistical evaluation of results can be performed using a number of methods including, but not limited to: the students T test, the two sided T test, pearson rank sum analysis, hidden markov model analysis, analysis of q-q plots, principal component analysis, one way analysis of variance (ANOVA), two way ANOVA, and the like. Statistical evaluation can be performed by the classifier or trained algorithm.

The methods disclosed herein may include extracting and analyzing protein or nucleic acid (RNA or DNA) from one or more samples from a subject. Nucleic acid can be extracted from the entire sample obtained or can be extracted from a portion. In some cases, the portion of the sample not subjected to nucleic acid extraction may be analyzed by cytological examination or immuno-histochemistry. Methods for RNA or DNA extraction from biological samples can include for example phenol-chloroform extraction (such as guanidinium thiocyanate phenol-chloroform extraction), ethanol precipitation, spin column-based purification, or others.

General methods for determining gene expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immuno-histochemistry, blotting, sequencing, RNA sequencing, DNA sequencing (e.g., sequencing of complementary deoxyribonucleic acid (cDNA) obtained from RNA); next generation (NextGen) sequencing, nanopore sequencing, pyrosequencing, or Nanostring sequencing. Gene expression product levels may be normalized to an internal standard such as total messenger ribonucleic acid (mRNA) or the expression level of a particular gene. There can be a specific difference or range of difference in gene expression between samples being compared to one another, for example a sample from a subject and a reference sample. The difference in gene expression level can be at least 5%, 10%, 15%, 20%, 25%, 30%, 35%, 40%, 45% or 50% or more. In some cases, the difference in gene expression level can be at least 2, 3, 4, 5, 9, 10 fold or more.

RNA Sequencing can produce two or more feature spaces such as counts of gene expression and presence of sequence variants of a particular sample. For example, RNA sequencing measures variants in genes expressed in a specific tissue or specific sample, such as a thyroid tissue or thyroid nodule. Next generation sequence can provide gene expression level data of a particular sample. Sequencing results, such as RNA sequencing and Next generation sequencing results, can be entered into a classifier that can combine unique feature spaces to determine the risk of occurrence of a disease with higher accuracy than using a single feature space. The classifier or trained algorithm can include algorithms that have been developed using a reference set of known malignant, benign, and normal samples. The classifier or trained algorithm can include algorithms that have been developed using a reference set of known low-risk, medium-risk, and high-risk samples.

### Markers for Array Hybridization, Sequencing, Amplification

Suitable reagents for conducting array hybridization, nucleic acid sequencing, nucleic acid amplification or other amplification reactions include, but are not limited to, DNA polymerases, markers such as forward and reverse primers, deoxynucleotide triphosphates (dNTPs), and one or more buffers. Such reagents can include a primer that is selected for a given sequence of interest, such as the one or more genes of the first set of genes and/or second set of genes.

In such amplification reactions, one primer of a primer pair can be a forward primer complementary to a sequence of a target polynucleotide molecule (e.g. the one or more genes of the first or second sets) and one primer of a primer pair can be a reverse primer complementary to a second sequence of the target polynucleotide molecule and a target locus can reside between the first sequence and the second sequence.

The length of the forward primer and the reverse primer can depend on the sequence of the target polynucleotide (e.g. the one or more genes of the first or second sets) and the target locus. In some cases, a primer can be greater than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or about 100 nucleotides in length. As an alternative, a primer can be less than about 100, 95, 90, 85, 80, 75, 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or about nucleotides in length. In some cases, a primer can be about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, about 20 to about 60, about 20 to about 80, or about 20 to about 100 nucleotides in length.

Primers can be designed according to known parameters for avoiding secondary structures and self-hybridization, such as primer dimer pairs. Different primer pairs can anneal and melt at about the same temperatures, for example, within 1° C., 2° C., 3° C., 4° C., 5° C., 6° C., 7° C., 8° C., 9° C. or 10° C. of another primer pair.

The target locus can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends of the plurality of template polynucleotides.

The markers (i.e., primers) for the methods described can be one or more of the same primer. In some instances, the markers can be one or more different primers such as about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more different primers. In such examples, each primer of the one or more primers can comprise a different target or template specific region or sequence, such as the one or more genes of the first or second sets.

The one or more primers can comprise a fixed panel of primers. The one or more primers can comprise at least one or more custom primers. The one or more primers can comprise at least one or more control primers. The one or more primers can comprise at least one or more housekeeping gene primers. In some instances, the one or more custom primers anneal to a target specific region or complements thereof. The one or more primers can be designed to amplify or to perform primer extension, reverse transcription, linear extension, non-exponential amplification, exponential amplification, PCR, or any other amplification method of one or more target or template polynucleotides.

Primers can incorporate additional features that allow for the detection or immobilization of the primer but do not alter a basic property of the primer (e.g., acting as a point of initiation of DNA synthesis). For example, primers can comprise a nucleic acid sequence at the 5′ end which does not hybridize to a target nucleic acid, but which facilitates cloning or further amplification, or sequencing of an amplified product. For example, the sequence can comprise a primer binding site, such as a PCR priming sequence, a sample barcode sequence, or a universal primer binding site or others.

A universal primer binding site or sequence can attach a universal primer to a polynucleotide and/or amplicon. Universal primers can include −47F (M13F), alfaMF, AOX3′, AOX5′, BGHr, CMV-30, CMV-50, CVMf, LACrmt, lamgda gt10F, lambda gt 10R, lambda gt11F, lambda gt11R, M13 rev, M13Forward(-20), M13Reverse, male, p10SEQPpQE, pA-120, pet4, pGAP Forward, pGLRVpr3, pGLpr2R, pKLAC14, pQEFS, pQERS, pucU1, pucU2, reversA, seqIREStam, seqIRESzpet, seqori, seqPCR, seqpIRES−, seqpIRES+, seqpSecTag, seqpSecTag+, seqretro+PSI, SP6, T3-prom, T7-prom, and T7-termInv. As used herein, attach can refer to both or either covalent interactions and noncovalent interactions. Attachment of the universal primer to the universal primer binding site may be used for amplification, detection, and/or sequencing of the polynucleotide and/or amplicon.

### Uses of Risk Determination

Results of the classifier, such as a risk of disease occurrence or data from methods disclosed herein, such as gene expression levels or sequence variant data can be entered into a database for access by representatives or agents of a molecular profiling business, an individual, a medical professional, or insurance provider. A computer or algorithmic analysis of the data can be provided automatically. Results can be presented as a report on a computer screen or as a paper record. Results can be uploaded, in some cases automatically, to a database or remote server. The report can include, but is not limited to, such information as one or more of the following: suitability of the original sample, the name and/or number of genes differentially expressed, the name and/or number of genes with sequence variants, the types of sequence variants, the expression level of genes differentially expressed, a numerical classifier score, a diagnosis for the subject, a statistical confidence for the diagnosis, a risk of occurrence of the disease, indicated therapies, or any combination thereof.

A subject may be monitored at a single time point or over multiple time points using the methods described herein. For example, a subject may be diagnosed with a disease such as cancer or a genetic disorder using the methods described herein. In some cases, this initial diagnosis may not involve the use of the methods described herein. The subject having a positive disease diagnosis, such as thyroid cancer, may then be prescribed a therapeutic intervention such as a thyroidectomy or to begin a drug regime, such as chemotherapy. The results of the therapeutic intervention may be monitored on an ongoing basis by using the methods described herein to detect the efficacy of the therapeutic intervention. In another example, a subject whom otherwise does not have cancer may be diagnosed with a risk of occurrence of cancer and may be monitored on an ongoing basis by the methods described herein to detect any changes in the state of their health status to determine whether cancer may become present at a later point in time or to influence the frequency of which to perform screening methods.

The methods as described herein may also be used to ascertain the potential efficacy of a specific therapeutic intervention prior to administering to a subject. For example, a subject may be diagnosed with cancer. The methods as described herein may indicate high levels of a gene expression in a gene product known to be involved in cancer malignancy, such as for example the RAS oncogene. A sample from the subject having the high levels may be obtained and cultured in vitro. The application of various inhibitors of the aberrantly activated or dysregulated pathway, or drugs known to inhibit the activity of the pathway may then be tested against the tumor cells of the sample for growth inhibition. Molecular profiling may also be used to monitor the effect of these inhibitors on for example down-stream targets of the implicated pathway. Molecular profiling may also be used to predict the efficacy of these inhibitors.

The methods described herein may be used as a research tool to identify new markers for diagnosis of a disease such as cancer; to monitor the effect of drugs or candidate drugs on samples such as tumor cells, cell lines, tissues, or organisms; or to uncover new pathways for disease progression or repression such as cancer oncogenesis and/or tumor suppression.

The methods described herein can provide: 1) gene expression analysis of samples containing low amount and/or low quality of nucleic acid; 2) a significant reduction of false positives and false negatives, 3) a determination of the underlying genetic, metabolic, or signaling pathways responsible for a resulting pathology, 4) the ability to assign a statistical probability to the accuracy of the diagnosis of disease such as genetic disorders, 5) the ability to resolve ambiguous results, 6) the ability to distinguish between sub-types of a disease such as cancer, and 7) the ability to distinguish between a low risk of occurrence of a disease and a medium-to-high risk of occurrence of a disease.

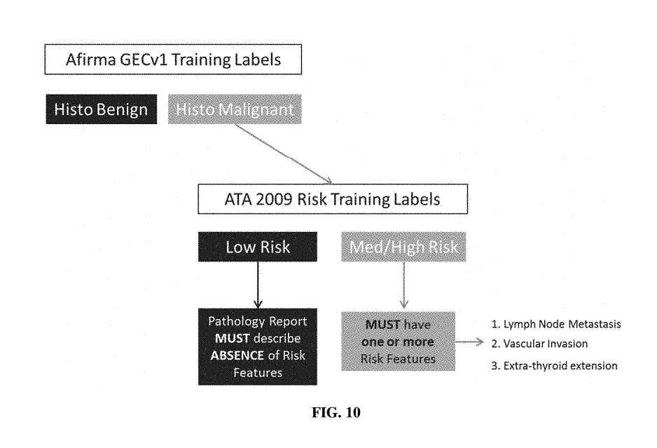

Predication may rely on accurate training labels. For example, as shown in FIG. 10, samples labeled or classified as histologically malignant in an Afirma Gene Expression Classifier (GEC) version 1, are further labeled or classified using the American Thyroid Association (ATA) staging system as either low risk of occurrence or medium/high risk of occurrence. For a sample to be labelled as a low risk of occurrence, a histopathology report may describe absence of one or more risk features. For a sample to be labelled as a medium/high risk of occurrence, a histopathology report may describe one or more risk features as being positively present. A risk feature may be a lymph node metastasis, a vascular invasion, an extra-thyroid extension, or any combination thereof.

A risk classifier may be trained using a single tissue sample comprising a specific subtype of cancer, for example, a tissue sample comprising papillary thyroid carcinoma (PTC). In some cases, a risk classifier is trained using a single tissue sample comprising two, three, four, or more subtypes of cancer, for example, PTC, LCT, HA, and FC. In some cases, a risk classifier may be trained using more than one tissue sample, for example two tissue samples, wherein the two tissue samples comprising two, three, four, or more subtypes of cancer, for example, PTC, LCT, and FC.

### Kits

The disease diagnostic business, molecular profiling business, pharmaceutical business, or other business associated with patient healthcare may provide a kit for performing the determining the risk of occurrence of a disease. The kit may include a classifier, a sample cohort for training the algorithm, and a list of genes for each feature space, such as a first set of genes and second set of genes. In some cases, the kit may include a classifier and a list of genes for each feature space. The kit may be a general kit for all disease types. The kit may be a specific kit for a specific disease such as cancer, or a specific kit to a disease subtype such as thyroid cancer. The kit may provide a classifier that has already been trained used a sample cohort not provided in the kit. The kit may provide periodic updates of sample cohorts or lists of genes for feature spaces to use with the classifier. The kit may provide software to automate a summary of results that can be reported or displayed or downloaded by the medical professional and/or entered into a database. The summary of results can include any of the results disclosed herein, including recommendations of treatment options for the patient and risk occurrence of a disease. The kit may also provide a unit or device for obtaining a sample from a subject (e.g., a device with a needle coupled to an aspirator). The kit may also provide instructions for performing methods as disclosed herein, and include all necessary buffers and reagents for RNA sequencing and next generation (NextGen) sequencing. The kit may also include instructions for analyzing the results. Such instructions may include directing the user to software (e.g., software with a trained algorithm) and databases for analyzing the results.

### Computer Control Systems

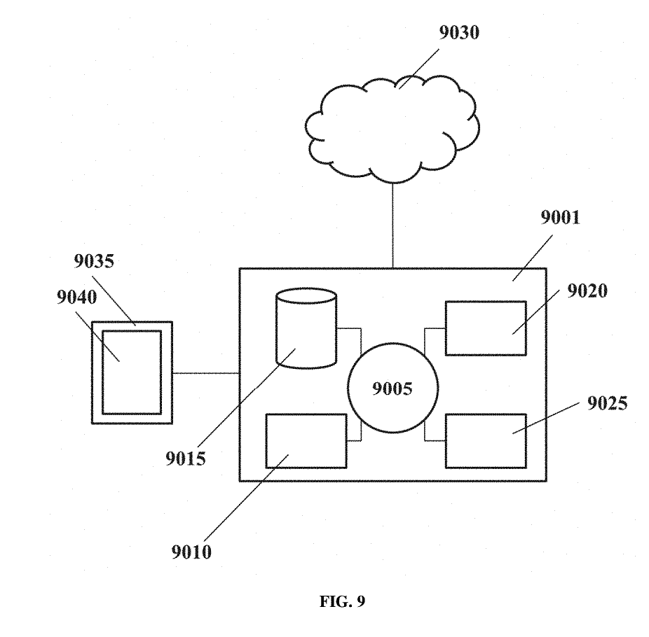

The present disclosure provides computer control systems that are programmed to implement methods of the disclosure. FIG. 9 shows a computer system 9001 that is programmed or otherwise configured to implement the methods provided herein. The computer system 9001 can regulate various aspects of stratifying risk of occurrence of disease of the present disclosure, such as, for example, running a classifier or training algorithm and reporting the stratified risk of occurrence. The computer system 9001 can be an electronic device of a user or a computer system that is remotely located with respect to the electronic device. The electronic device can be a mobile electronic device.

The computer system 9001 includes a central processing unit (CPU, also “processor” and “computer processor” herein) 9005, which can be a single core or multi core processor, or a plurality of processors for parallel processing. The computer system 9001 also includes memory or memory location 9010 (e.g., random-access memory, read-only memory, flash memory), electronic storage unit 9015 (e.g., hard disk), communication interface 9020 (e.g., network adapter) for communicating with one or more other systems, and peripheral devices 9025, such as cache, other memory, data storage and/or electronic display adapters. The memory 9010, storage unit 9015, interface 9020 and peripheral devices 9025 are in communication with the CPU 9005 through a communication bus (solid lines), such as a motherboard. The storage unit 9015 can be a data storage unit (or data repository) for storing data. The computer system 9001 can be operatively coupled to a computer network (“network”) 9030 with the aid of the communication interface 9020. The network 9030 can be the Internet, an internet and/or extranet, or an intranet and/or extranet that is in communication with the Internet. The network 9030 in some cases is a telecommunication and/or data network. The network 9030 can include one or more computer servers, which can enable distributed computing, such as cloud computing. The network 9030, in some cases with the aid of the computer system 9001, can implement a peer-to-peer network, which may enable devices coupled to the computer system 9001 to behave as a client or a server.

The CPU 9005 can execute a sequence of machine-readable instructions, which can be embodied in a program or software. The instructions may be stored in a memory location, such as the memory 9010. The instructions can be directed to the CPU 9005, which can subsequently program or otherwise configure the CPU 9005 to implement methods of the present disclosure. Examples of operations performed by the CPU 9005 can include fetch, decode, execute, and writeback.

The CPU 9005 can be part of a circuit, such as an integrated circuit. One or more other components of the system 9001 can be included in the circuit. In some cases, the circuit is an application specific integrated circuit (ASIC).

The storage unit 9015 can store files, such as drivers, libraries and saved programs. The storage unit 9015 can store user data, e.g., user preferences and user programs. The computer system 9001 in some cases can include one or more additional data storage units that are external to the computer system 9001, such as located on a remote server that is in communication with the computer system 9001 through an intranet or the Internet.

The computer system 9001 can communicate with one or more remote computer systems through the network 9030. For instance, the computer system 9001 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 9001 via the network 9030.

Methods as described herein can be implemented by way of machine (e.g., computer processor) executable code stored on an electronic storage location of the computer system 9001, such as, for example, on the memory 9010 or electronic storage unit 9015. The machine executable or machine readable code can be provided in the form of software. During use, the code can be executed by the processor 9005. In some cases, the code can be retrieved from the storage unit 9015 and stored on the memory 9010 for ready access by the processor 9005. In some situations, the electronic storage unit 9015 can be precluded, and machine-executable instructions are stored on memory 9010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 9001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 9001 can include or be in communication with an electronic display 9035 that comprises a user interface (UI) 9040 for providing, for example, an output or readout of the classifier or trained algorithm. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 9005. The algorithm can, for example, stratifying risk of occurrence of a disease or classifying a sample as benign, malignant, suspicious, or non-diagnostic.

### Example 1: Risk Stratification of Sample Using Risk Classifier

Current risk adapted approaches to initial management of thyroid cancer is based upon post-operative classification of subjects as either high-intermediate risk or low risk of occurrence utilizing the 2009 American Thyroid Association staging system (ATA). While this anatomic staging system can be clinically useful, it cannot be accurately assessed prior to thyroidectomy, and it cannot include any molecular predictors of subject outcome. This study determines if transcriptional data obtained during diagnostic fine needle aspiration (FNA) of malignant thyroid nodules could be used to augment risk stratification prior to thyroid surgery.

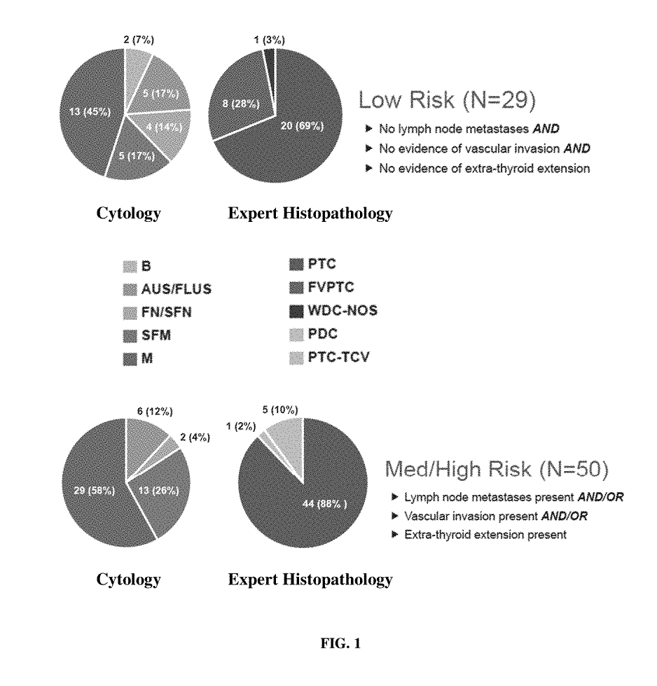

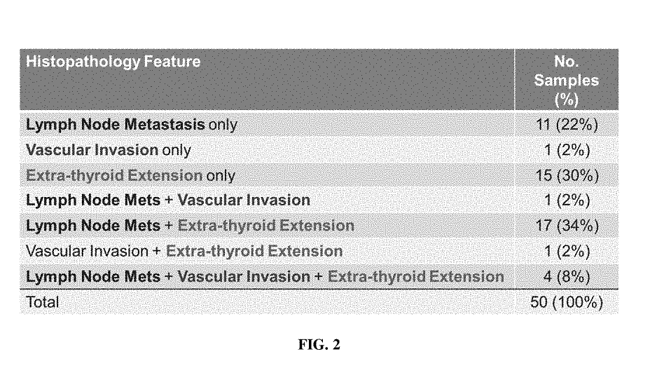

FNA material from samples is preoperatively collected (n=79) and post-surgically diagnosed by a panel of experts as papillary thyroid carcinoma (PTC), including classic histologic subtypes (FIG. 1 and FIG. 2). Each patient is categorized as either “low risk” or “medium-to-high risk” using established guidelines for occurrence risk stratification. Genome-wide RNA Sequence (RNASeq) data (80 million reads per sample) is obtained and supervised learning is used to train classifiers; including Support Vector Machine (SVM), Random Forest (RF), penalized logistic regression (PLR), and an ensemble of the three. Classifier performance is measured using 10-fold cross-validation on the same sample cohort.

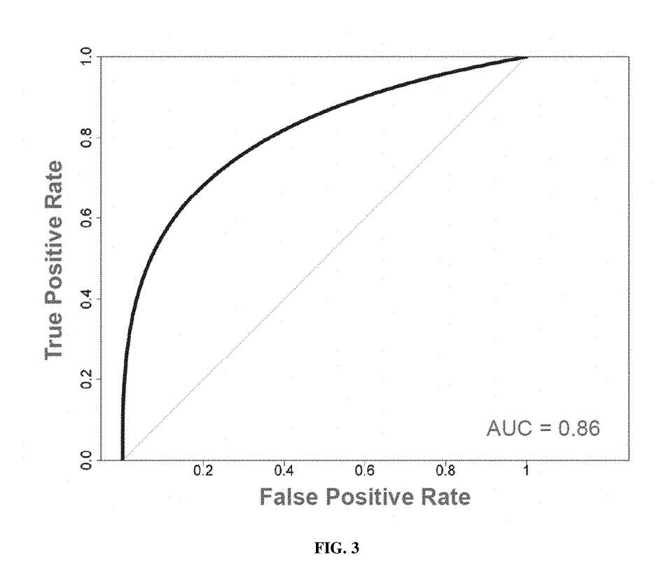

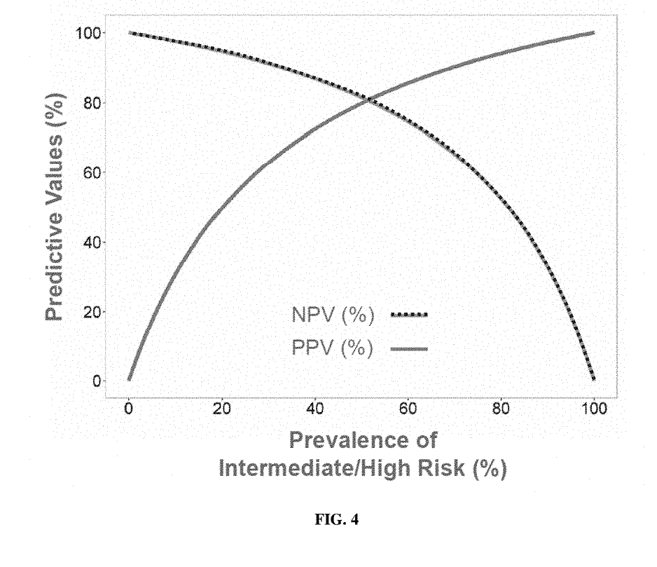

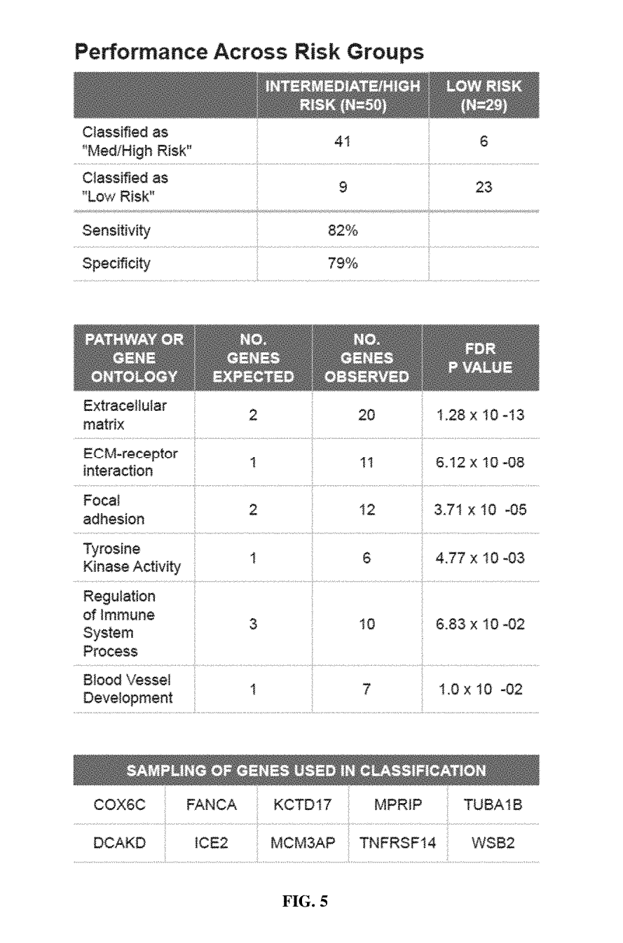

Classifiers are built using 320 genes and open source software DESeq models that controlled for BRAF gene status. Maximum classification performance of “low risk” vs. “medium-to-high risk” is observed for an support vector machine (SVM) classifier with a maximal area under the receiver operating characteristic (ROC) curve (AUC) of 0.86 (FIG. 3 and FIG. 4). All classifiers achieve similar AUCs: RF 0.82, PLR 0.82, and ensemble 0.84. Genes discovered to be useful in classification belong to a variety of transmembrane signaling pathways including ECM-receptor interaction, focal adhesion, and cell adhesion molecules (FIG. 5). The classifiers evaluated use a threshold that optimized total accuracy, favoring neither sensitivity nor specificity. When applied to the sample cohort, the support vector machine (SVM) classifier correctly identifies 79.3% (23/29) of American Thyroid Association (ATA) low risk tumors and 82.0% (41/50) of ATA medium-to-high risk tumors (FIG. 5).

### Example 2: Cross-Validation Model

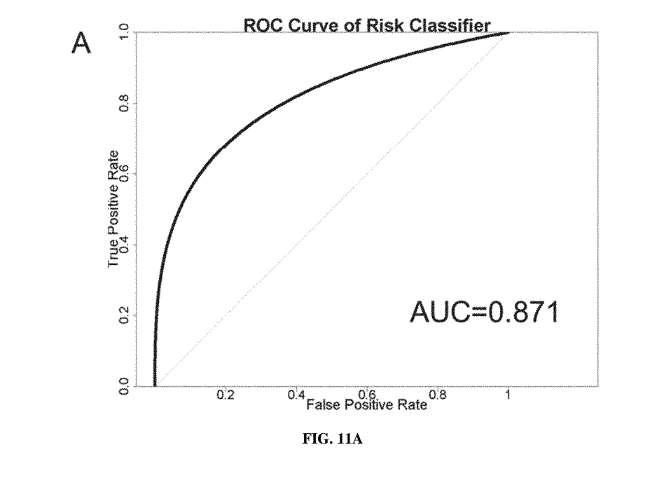

Indeterminate thyroid nodules are tested employing a Gene Expression Classifier (GEC) with mutational panels to determine whether pre-operative risk stratification is augmented by employing machine learning. FIG. 10 is a flow diagram showing the determination of training labels. Afirma GEC version 1 training labels are employed to distinguish between histological benign samples and histologically malignant samples. The histologically malignant samples are further distinguished between low risk of occurrence and medium/high risk of occurrence using the American Thyroid Association (ATA) Risk training labels. Medium/high risk features include lymph node metastasis, vascular invasion, extra-thyroid extension, or any combination thereof. The risk training sample cohort is shown in FIG. 1. The percent of samples having the medium/high risk of occurrence histological features is shown in FIG. 2. A 10-fold cross-validation is performed to evaluate the Area Under the Curves (AUCs) for different learning models including a linear support vector machine (SVM), Random Forest, GLMNet, and Ensemble Classifier. In this example, the best model is the Ensemble Classifier which has an AUC of 0.871 (as shown in FIG. 11A), a sensitivity of 86% (as shown in FIG. 11B), and a specificity of 86% (as shown in FIG. 11B), a positive predictive value (PPV) of 91.3%, and a negative predictive value (NPV) of 78.3%. The initial feature space is 850 initial features, including 50 counts and 800 variants. The best performance is using 240 combined features. The top features from the variants selected by the classifier in every fold are shown in FIG. 12. The top features from the counts selected 8 to 10 times by the classifier in 10 folds are shown in FIG. 13.

### Example 3: Mutational Analysis

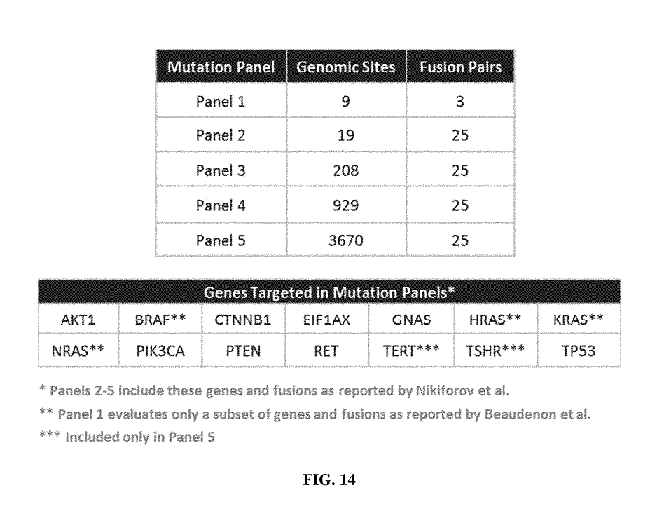

Fine needle aspirate (FNA) samples (n=81) are collected and post-surgically diagnosed by a panel of experts as malignant (papillary thyroid carcinoma (PTC), multifocal papillary thyroid carcinoma (mPTC), follicular variant of papillary thyroid carcinoma (FVPTC), papillary thyroid carcinoma with tall-cell features (PTC-TCV), medullary thyroid cancer (MTC), well-differentiated carcinoma-not otherwise specified (WDC-NOS), hepatocellular cancer (HCC), follicular cancer (FC)) or benign (benign familial neutropenia (BFN), fibroadenoma (FA), hepatocellular adenoma (HCA), hyalinizing trabecular adenoma (HTA), Leydig cell tumour (LCT)). Surgical tissue samples (n=57) having histopathology truth are also analyzed. A consecutive series of indeterminate FNAs (n=101) from a Clinical Laboratory Improvement Amendments (CLIA) lab without histopathology are also analyzed. Samples are subjected to Next Generation Sequencing (NGS) and 14 genes (FIG. 14) are evaluated with increasing numbers of interrogated genomic sites and fusion pairs in the five different mutational panels. As shown in FIG. 14, the upper table indicates the number of genomic sites and the number of fusion pairs for each of the five mutation panels. Mutation panel 1 is comprised of 9 genomic sites and 3 fusion pairs. Mutation panel 2 is comprised of 19 genomic sites and 25 fusion pairs. Mutation panel 3 is comprised of 208 genomic sites and 25 fusion pairs. Mutation panel 4 is comprised of 929 genomic sites and 25 fusion pairs. Mutation panel 5 is comprised of 3670 genomic sites and 25 fusion pairs. The lower table of FIG. 14 shows the 14 genes targeted in one or more of the mutation panels.

Several filters are applied to score the data. Samples are scored negative when no fusions or point mutations are present. Samples are scored positive if at least one fusion or point mutation is detected, except for guanine nucleotide binding protein, alpha stimulating (GNAS) mutations, markers of which are considered to be markers of benignity.

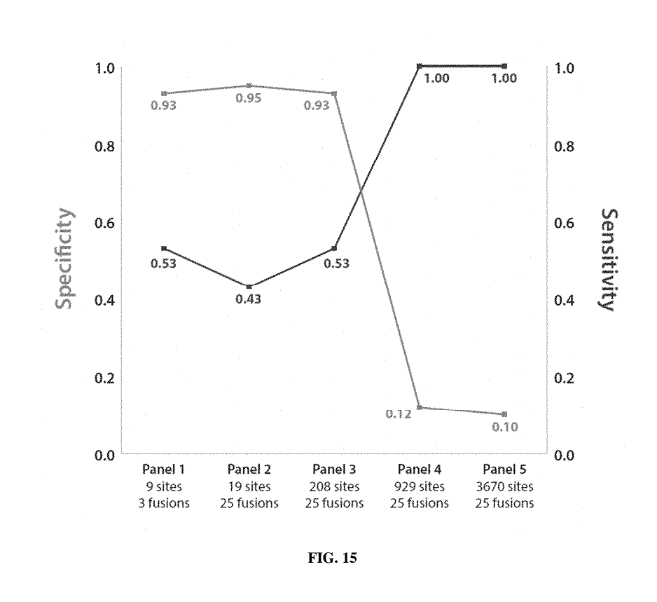

Sensitivity to detect malignancy improves in all sample cohorts with increasing number of loci. Specificity shows the opposite trend, decreasing in all sample cohorts with increasing number of loci. In FNA samples in FIG. 15, the smallest 9 site panel renders a sensitivity of 53% and a specificity of 93%. The largest panel (3670 sites) in FIG. 15 renders a sensitivity of 100% and a specificity of 10%.

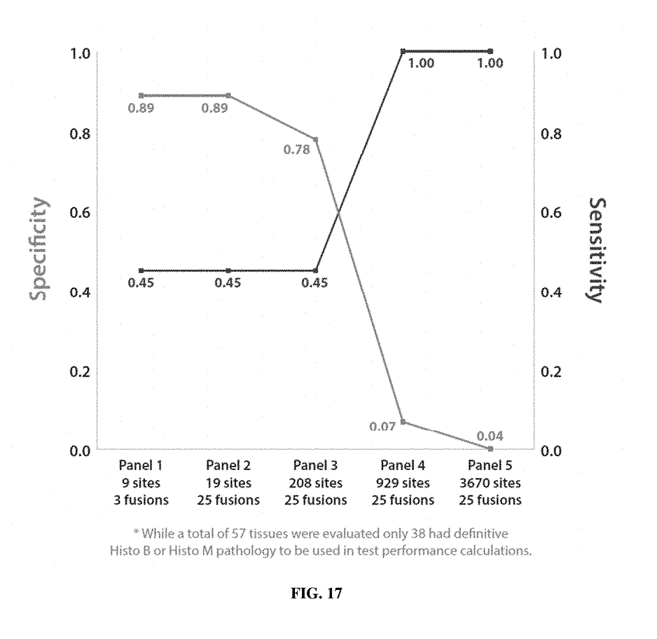

In surgical tissues (n=38) in FIG. 17, a similar trend is observed. A total of 57 tissues are evaluated. However, only 38 tissues have definitive histologically benign or histologically malignant pathology to be used in the test performance calculations. In the smallest 9 site panel of FIG. 17, 89% specificity is associated with 45% sensitivity. In the densest panel (3670 sites) of FIG. 17, a sensitivity of 100% is associated with 0% specificity.

Overall, the two larger panels of FIG. 15 and FIG. 17 wrongly called 87-90% of histology benign FNAs as malignant, while the two smaller panels of FIG. 15 and FIG. 17 miss 48-58% of known cancers. The frequency of mutations and fusions in the CLIA FNA samples across the five panels is 13%, 4%, 21%, 89% and 92%, respectively. Sensitivity gained by detecting increasingly larger numbers of point mutations and fusions come at the cost of specificity and run the risk of overcalling malignancy in truly benign samples.

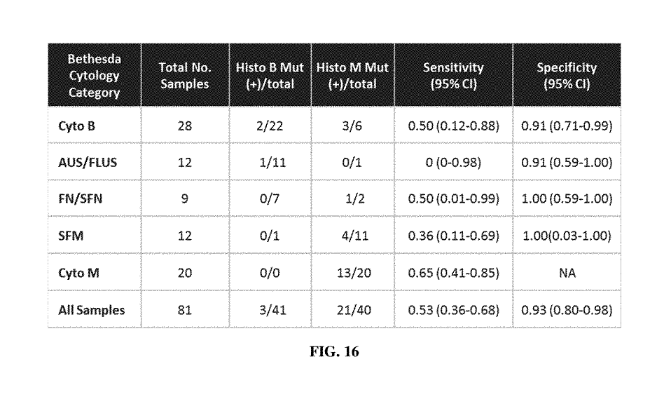

The mutation performance by cytology in panel 3, having 208 sites, is shown in FIG. 16. The groups are divided by the Bethesda Cytology Category which includes cytologically benign (Cyto B), Atypia of Undetermined Significance/Follicular Lesion of Underdetermined Significance (AUS/FLUS), follicular neoplasm/suspicious for follicular neoplasm (FN/SFN), suspicious for malignancy (SFM), cytologically malignant (Cyto M), and all the samples. Several parameters including the total number of samples, the number of histologically benign mutations per total, the number of histologically malignant mutations per total, the sensitivity, the specificity are shown for each group in FIG. 16.

A graphical representation of mutation frequency observed for the CLIA FNA samples is shown in FIG. 18A. Mutation positive samples (Panel 3) are indicated in a dark gray color. GNAS positive nodules are indicated in a light gray color. Percent mutation frequency is subdivided into different groups including an overall group, an AUS/FLUS group, and an FN/SFN group. FIG. 18B shows a table of genes and mutations that were detected with panel 3 in the various subgroups also shown in FIG. 18A.

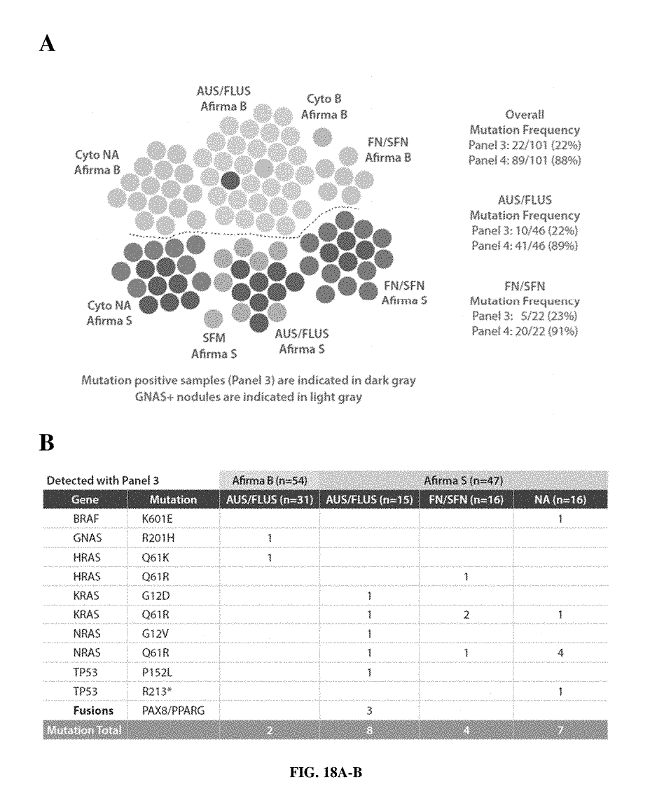

A graphical representation of mutation frequency observed for the FNA samples is shown in FIG. 19A. Mutation positive nodules (Panel 3) are indicated in dark gray. Nodules are depicted size proportional with the smallest nodule=1 centimeter (cm). Percent mutation frequency is subdivided into different groups including an overall group, a histologically malignant group, and a histologically benign group. FIG. 19B shows a table of genes and mutations that are detected with panel 3 in the various subgroups also shown in FIG. 19A.

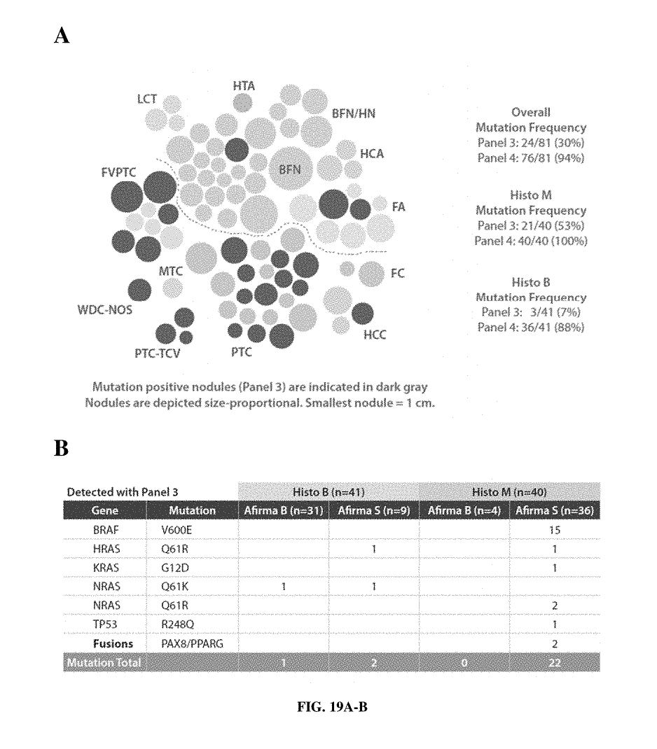

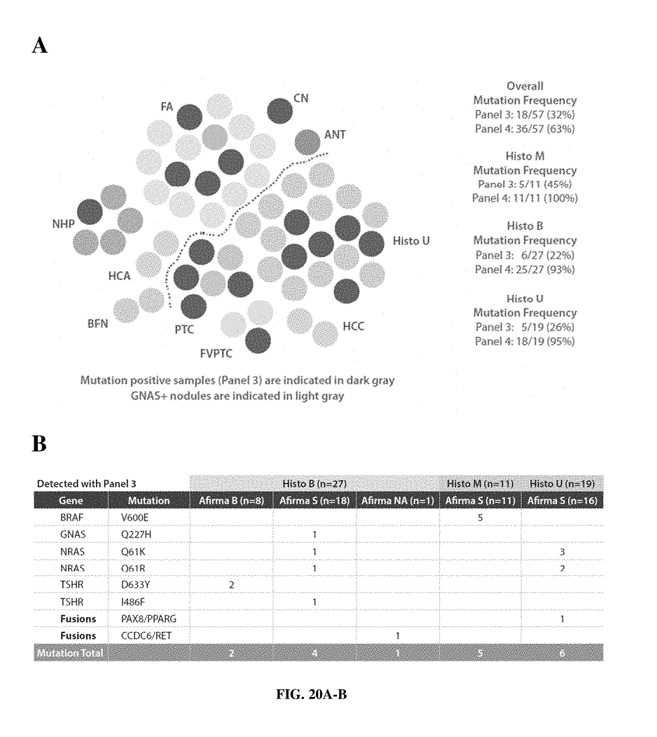

A graphical representation of mutation frequency observed for the tissue samples is shown in FIG. 20A. Mutation positive samples (Panel 3) are indicated in dark gray. GNAS positive nodules are indicated in light gray. Percent mutation frequency is subdivided into different groups including an overall group, a histologically malignant group, a histologically benign group, and a histologically unsatisfactory or nondiagnostic group. FIG. 20B shows a table of genes and mutations that are detected with panel 3 in the various subgroups also shown in FIG. 20A.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-52. (canceled)

53. A method for analyzing a sample from a subject, comprising:
(a) subjecting said sample to cytological analysis to determine that said sample is ambiguous or suspicious;
(b) upon identifying that said sample is ambiguous or suspicious, obtaining an expression level of transcripts from said sample, which expression level of transcripts correspond to one or more genes of a first set of genes;
(c) subjecting nucleic acid molecules from said sample to sequencing to generate a plurality of nucleic acid sequences;
(d) processing said plurality of nucleic acid sequences to determine (i) a presence of a nucleic acid sequence corresponding to a gene of a second set of genes in said sample, and (ii) a presence of one or more sequence variants with respect to a given gene of said second set of genes; and
(e) determining a risk of occurrence of a disease in said subject based on said expression level of transcripts of (b) and said presence of one or more sequence variants of (d).

54. The method of claim 53, further comprising comparing said expression level of transcripts from (b) and said presence of said one or more sequence variants from (d) to a reference set.

55. The method of claim 53, wherein (c) comprises generating cDNA from said nucleic acid molecules and subsequently subjecting said cDNA to nucleic acid sequencing.

56. The method of claim 53, wherein said disease is cancer.

57. The method of claim 53, further comprising, prior to (a), obtaining said sample from said subject.

58. The method of claim 53, further comprising comparing said nucleic acid sequence of (d) to a reference sequence to identify said presence of one or more sequence variants.

59. The method of claim 53, wherein said risk of occurrence of said disease includes (i) a risk of recurrence of said disease in said subject or (ii) a risk of metastasis in said subject.

60. The method of claim 54, wherein said reference set comprises tissue samples obtained from at least 25 subjects having been diagnosed with said disease.

61. The method of claim 53, wherein (e) occurs pre-operatively.

62. The method of claim 53, wherein (e) occurs prior to said subject having a positive disease diagnosis.

63. The method of claim 53, wherein (e) further comprises stratifying said risk of occurrence into a low risk of occurrence or a medium-to-high risk of occurrence, wherein said low risk of occurrence has a probability of occurrence between about 50% and about 80% and wherein said medium-to-high risk of occurrence has a probability of occurrence between about 80% and 100%.

64. The method of claim 63, wherein said stratifying has an accuracy of at least about 80%.

65. The method of claim 63, wherein said stratifying has a specificity of at least about 80%.

66. The method of claim 54, wherein said comparing is performed using a computer processor that is programmed with a trained algorithm to (i) compare said expression level of transcripts from (b) and said presence of said one or more sequence variants from (d) to said reference set and (ii) determine said risk of occurrence of said disease in said subject.

67. The method of claim 66, wherein said trained algorithm is trained with a training set of samples comprising fine needle aspirate (FNA) samples.

68. The method of claim 66, further comprising applying one or more filters, one or more wrappers, one or more embedded protocols, or any combination thereof to said trained algorithm.

69. The method of claim 68, further comprising applying said one or more filters to said trained algorithm and wherein said one or more filters comprises a t-test, an analysis of variance (ANOVA) analysis, a Bayesian framework, a Gamma distribution, between-within class sum of squares test, a rank products method, a random permutation method, a threshold number of misclassification (TNoM), a bivariate method, a correlation based feature selection (CFS) method, a minimum redundancy maximum relevance (MRMR) method, a Markov blanket filter method, an uncorrelated shrunken centroid method, or any combination thereof.

70. The method of claim 53, wherein a sequence variant of said one or more sequence variants comprise one or more of a point mutation, a fusion gene, a substitution, a deletion, an insertion, an inversion, a conversion, a translocation, or any combination thereof.

71. The method of claim 53, wherein said first set of genes or said second set of genes is less than about 15 genes.

72. The method of claim 53, wherein said first set of genes or said second set of genes is less than about 75 genes.

73. The method of claim 53, wherein said first set of genes or said second set of genes is between about 50 and about 400 genes.

74. The method of claim 53, wherein said sequencing of (c) comprises enriching for one or more genes of said second set of genes or variants thereof.

75. The method of claim 53, wherein said sample comprises a thyroid tissue sample.

76. The method of claim 53, wherein said first set of genes and said second set of genes are different.

77. The method of claim 53, wherein said obtaining in (b) comprises assaying for said expression level of transcripts corresponding to each of said one or more genes of said first set of genes.

78. The method of claim 53, wherein said obtaining in (b) comprises employing array hybridization, nucleic acid sequencing or nucleic acid amplification using probes that are selective for said one or more genes of said first set of genes.

79. The method of claim 53, wherein said sequencing in (c) employs probes that are selective for said one or more genes of said second set of genes.

80. The method of claim 53, wherein said sample comprises a fine needle aspirate sample.

81. The method of claim 53, wherein said first set of genes is associated with said risk of occurrence of said disease in said subject.
